(19) United States
(12) Patent Application Publication
(10) Pub. No.: US 2014/0011706 A1
(43) Pub. Date: Jan. 9, 2014

(54) SURFACE DISPLAY OF POLYPEPTIDES CONTAINING A METAL PORPHYRIN OR A FLAVIN

(71) Applicants: Stephanie Schumacher, Duesseldorf (DE); Rita Bernhardt, Saarbruecken (DE); Frank Hannemann, Saarbruecken (DE); Joachim Jose, Duesseldorf (DE)

(72) Inventors: Stephanie Schumacher, Duesseldorf (DE); Rita Bernhardt, Saarbruecken (DE); Frank Hannemann, Saarbruecken (DE); Joachim Jose, Duesseldorf (DE)

(73) Assignee: Autodisplay Biotech GMBH, Duesseldorf (DE)

(21) Appl. No.: 13/825,837

(22) PCT Filed: Sep. 22, 2011
(86) PCT No.: PCT/EP11/66517

§ 371 Date: Jun. 3, 2013

(30) Foreign Application Priority Data
Sep. 23, 2010 (EP) 10179060.8

(51) Int. Cl. C12N 9/00

(52) U.S. Cl. CPC C12N 9/0073
USPC 506/11; 435/69.1; 435/252.33; 435/170; 435/69.4; 435/29; 435/262.5; 435/189

(57) ABSTRACT

The present invention relates to a method for the display of recombinant functional polypeptides containing a prosthetic group selected from metal porphyrin and flavin containing groups on the surface of a host cell using the transporter domain of an autotransporter.

The present invention relates to a method for functionally displaying a recombinant polypeptide containing a prosthetic group on the surface of a host cell, wherein the prosthetic group is selected from metal porphyrins and flavins.

Over the past 30 years, it has become clear that enzymes hold great potential for industry. They are most remarkable biomolecules because of their extraordinary specificity and catalytic power [1]. The specificity and (enantio- and regio-)selectivity of certain enzymatic transformations makes them attractive for the production of fine chemicals and pharmaceutical intermediates. To date, more than 500 products are manufactured by enzymes. Well-known examples are ephedrine, aspartame and amoxicillin [2,3,4].

Cytochromes P450 enzymes have been discovered about 50 years ago and are ubiquitously distributed enzymes, which possess high complexity and display a broad field of catalytic activities. They are hemoproteins, which means, they contain a porphyrin ring system. The P450 enzyme family is involved in the biotransformation of drugs, the bioconversion of xenobiotics, the metabolism of chemical carcinogens, the biosynthesis of physiologically important compounds such as steroids, fatty acids, eicosanoids, fat-soluble vitamins, bile acids, the conversion of alkanes, terpenes, and aromatic compounds as well as the degradation of herbicides and insecticides [5]. Furthermore, there is a broad versatility of reactions catalysed by cytochromes P450, such as carbon hydroxylation, heteroatom oxygenation, dealkylation, epoxidation, aromatic hydroxylation, reduction, and dehalogenation.

Despite their very interesting features for industrial applications, the use of P450 enzymes for wide biotechnology needs is still limited, due to their difficulty in handling. With the exception of a few bacterial P450s, the vast majority needs a certain membrane contact or environment to fold into an active form. Within those membrane associated P450s, two classes can be identified: a mitochondrial and a microsomal. At the moment there are two different ways to use these enzymes for synthetic purposes. They are either purified after recombinant expression and reconstituted with an artificial membrane system, or they are expressed and used in a whole cell context. Both ways have their limitations. Reconstituted membrane vesicles with P450 enzymes are laborious to produce and they are absolutely not suited for industrial applications. Using whole cells with intrinsic P450s limits the set of substrates to be converted to those which are able to cross membranes [6].

Among other systems for the secretion of proteins in Gram-negative bacteria, the autotransporter pathway represents a solution of impressing simplicity. It is possible to transport a protein, regardless whether it is recombinant or the natural passenger, to the actual outer membrane, as long as its coding region lies between a typical signal peptide and a C-terminal domain called β-barrel. Based on these findings the autodisplay system has been developed by the use of the natural E. coli autotransporter protein AIDA-I (the adhesin involved in diffuse adherence) in an E. coli host background [9]. Autodisplay has been used for the surface display of random peptide libraries that were successfully screened for the identification of new enzyme inhibitors, and the display of functional enzymes like esterases, oxidoreductases and electron transfer proteins [10].

During the autodisplay of bovine adrenodoxine, which serves as an electron donor for mitochondrial p450s, two major observation were made [11,12]. First, it could be shown, that it is possible to incorporate an inorganic, prosthetic group into an apoprotein expressed by autodisplay at the cell surface by a simple titration step to yield a functional electron donor without loss of cell viability or cell integrity. And second, after external addition of the purified P450s CYP11B1 and CYP11A1, a functional whole cell biocatalyst was obtained for efficient synthesis of different steroids. Therefore the aim of the present invention is, to investigate, whether it is possible to autodisplay a P450 enzyme in a functional form on the surface of E. coli. This could provide a new expression platform for the highly interesting group of P450 enzymes with the perspective of being applicable in industrial processes.

### BACKGROUND

Cytochromes P450 are external monooxygenases. Monooxygenases (mixed function oxidases) catalyse the incorporation of a single atom of molecular oxygen into a substrate with the concomitant reduction of the other atom to water. Monooxygenases are divided into two classes: internal and external. Internal monooxygenases extract two reducing equivalents from the substrate to reduce one atom of dioxygen to water, whereas external monooxygenases utilize an external reductant. While initially the microsomal drug and xenobiotic-metabolising enzymes were referred to as mixed function oxidases, in more recent years the term monooxygenase became the widely accepted one.

Cytochromes P450 got their name from their character as hemoproteins as well as their unusual spectral properties displaying a typical absorption maximum of the reduced CO-bound complex at 450 nm: cytochrome stands for a hemoprotein, P for pigment and 450 reflects the absorption peak of the CO complex at 450 nm. The ability of reduced P450 to produce an absorption peak at 450 nm upon CO binding is still used for the estimation of the P450 content (Omura and Sato, 1964). The red shift of about 30 nm as observed in cytochromes P450 means that the distribution of electron density at the heme is significantly perturbed as compared to other cytochromes. It has been documented that the cause of this is the thiolate sulphur, which by means of a direct bond to the iron causes this effect. The Soret band (named after its discoverer) describes the absorption band of hemoproteins at about 380-420 nm.

Cytochrome P450 systems mainly catalyse the following reaction:

RH+O₂+NAD(P)H+H⁺→ROH+H₂O+NAD(P)⁺

They are involved in reactions as diverse as e.g. hydroxylation, N-, O- and S-dealkylation, sulphoxidation, epoxidation, deamination, desulphuration, dehalogenation, peroxidation, and N-oxide reduction. This diversity of catalysed reactions and, of course, the high amount of acceptable substrates is attractive for biotechnological application in particular when it can be transferred to industrial needs.

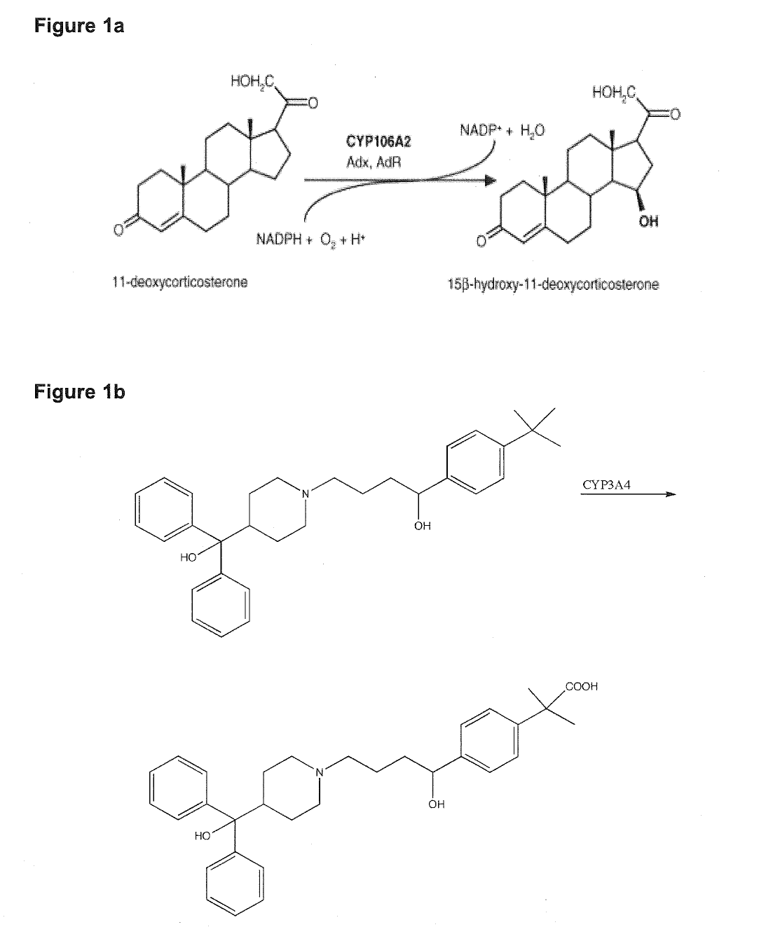

CYP106A2 is a bacterial steroid hydroxylase from Bacillus megaterium ATCC 13368. Since it is soluble and easy to express it has application for biotechnological purposes. Recently, it was possible to design a whole cell bioconversion system for steroids using a mixed system composed of the bovine mitochondrial electron transfer system AdR and Adx and the bacterial enzyme CYP106A2. This mixed P450 monooxygenase system was expressed in E. coli cells. Those successful experiments opened the door to facilitate the application of molecular evolution approaches in order to select mutants of the cytochrome with higher stability, activity, and changed regio- and stereo-specificity suitable to produce hydroxylated steroid derivatives using a biological transformation process [6]. CYP106A2 catalyzes as main reaction route the 15β-hydroxylation of several steroids, e.g. 11-deoxycorticosterone, testosterone, progesterone, and corticosterone (FIG. 1a) [7]. One disadvantage of this system is the limitation for substrates which are membrane permeable like corticosteroids. A successful expression via autodisplay could broaden the variety of substrates and therefore make the P450 monooxygenase system more valuable for future research.

CYP3A4 is the quantitative most important CYP-enzyme and involved in the oxidation of the largest range of substrates of all CYPs. In humans it is predominantly found in the liver and often allows prodrugs to be activated and absorbed. Inhibition or induction of CYP3A4 is a major problem in the daily clinical routine, since it often causes drug-drug interactions or side effects. Induction can lead to the fast inactivation of the applied drug and in consequence to plasma levels so low, that they do not have the desired therapeutic effect anymore. A commonly used CYP3A4 inductor is the anticonvulsant Carbamazepin. Inhibition instead can cause major intoxications due to plasma levels far beyond the therapeutic dose. On the other hand the capability of inhibition is used in the antiretroviral therapy to lower side effects and make it more bearable for patients. Ritonavir is given in a subtherapeutic dose to inhibit the enzyme and booster the effect of further antiretroviral drugs such as Lopinavir. A well-documented example is that of terfenadine, a nonsedating antihistamine (FIG. 1b). The oxidation of terfenadine is catalyzed very rapidly by CYP3A4 to its major metabolite fexofenadine which is responsible for the pharmacological activity. [8]

The E. coli outer membrane channel-tunnel protein TolC is involved in the exclusion of harmful substances such as antibiotics, dyes, organic solvents, and detergents. The crystal structure of the TolC protein recently has been determined. The TolC protein is composed of a transmembrane domain and a periplasmic domain and forms a homotrimer. The periplasmic barrel structure of TolC is connected to drug efflux pump proteins such as AcrB and AcrE, which are located on the inner membrane. Clamp proteins such as AcrA and ArcF link TolC and pump proteins in the periplasmic space. Pump proteins seem to transport toxic cytoplasmic or periplasmic substances into the extracellular space across the outer membrane via the TolC channel [26].

Porphyrins can act as photosensitizers. If porphyrins accumulate, they can be toxic, as the cells can become sensitive to near-UV irradiation. TolC is involved in porphyrin transport across the cell membrane and provides a mechanism to eliminate superfluous or/and toxic porphyrins. The TolC outer membrane channel-tunnel protein can function together with inner membrane efflux pump proteins. Therefore, an inner membrane pump(s) or exporter(s) is assumed to be involved in porphyrin exclusion in combination with TolC. Porphyrin(ogen) exclusion is considered as a two-step process. In this process, porphyrin(ogen)s are transported to the periplasm by a TolC-independent mechanism and then are transported across the outer membrane by the TolC-dependent efflux system [26].

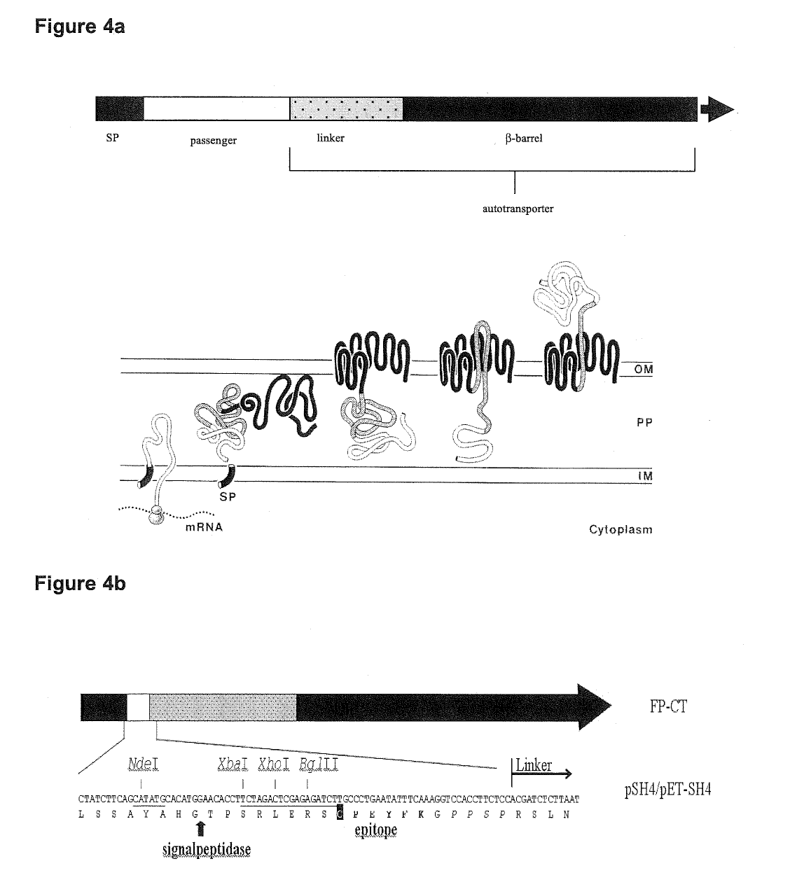

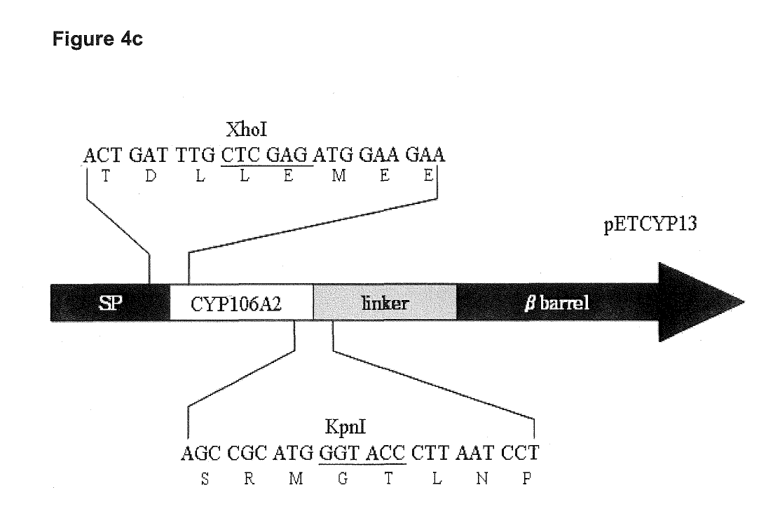

Autodisplay is based on the secretion mechanism of the autotransporter family or proteins [13]. A concept for this secretion mechanism was proposed concurrently with the first autotranspoter protein, IgA1protease from Neisseria gonorrhoeae (FIG. 4a) [14]. With the aid of a typical signal peptide, the precursor is transported across the inner membrane. Arrived in the periplasm, the C terminal part of the precursor forms a porin-like structure, a so-called β-barrel, within the outer membrane and through this pore the N terminally attached passenger (the actual protease) is translocated to the cell surface. To obtain full surface exposure of the passenger, a linker peptide is required in between the β-barrel and the passenger.

For the development of the autodisplay system the 3-barrel and the linker region of AIDA-I were combined in frame with the signal peptide of the cholera toxin β-subunit (CTB) and a strong constitutive promoter (P_{TK}) within a medium copy number plasmid backbone [15]. Into the linker regions used for autodisplay, protease cleavage sites for the sequence specific release of the passenger protein, as well as epitopes for detection by monoclonal antibodies were inserted. An antibody independent detection method, which requires only the addition of a single cysteine in the linker region, was developed for autodisplay and was named “Cystope tagging” [16,17]. A schematic description of the structure of a typical artificial autotransporter protein used for autodisplay is given in FIG. 4b. As mentioned above, the terminal step in autodisplay requires the translocation of the passenger through a size-limited pore formed by the β-barrel. This means that the passenger is not allowed to acquire a stable three dimensional conformation during transport to maintain a transport compatible state [18,19]. In case of stable folding, transport is blocked in the periplasm [19]. As a wide variety of passenger proteins with high biotechnological impact contain disulfide bridges and these bonds are normally formed in the periplasm of E. coli, a DsbA-negative mutant strain of E. coli (JK321) was constructed and shown to facilitate the autodisplay of such types of proteins as well [19]. In summary, the autodisplay system consists of vectors encoding various artificial autotransporter genes using the β-barrel from AIDA-I and different parts of its linkerregion. Dependent on the application, different modifications of the linker regions, various signal peptides under the control of inducible or constitutive promoters, mutant strains of E. coli supporting the transport and the surface display by the autotransporter pathway and detection methods are now available, that allow to follow surface translocation, preferentially independent of the protein domain used as a passenger. It is obvious, that autodisplay is restricted to Gram-negative bacteria i.e. E. coli or Salmonella as host organisms. Beyond this limitation, the autodisplay system has interesting activa. First, more than 100.000 active enzyme molecules can be displayed per single cell of E. coli without loss in cell integrity. Second, dimers or multimers can be formed spontaneously at the cell surface by subunits expressed from monomeric genes, which is a unique feature of this surface display system and due to the free motility of the anchoring motif, the β-barrel within the outer membrane. Third, EP 02718168 describes that anorganic prosthetic groups (e.g. 2Fe-2S) can be incorporated by a single step/one vial procedure without affecting cell viability, another feature that has not been described for any other surface display system so far. These features have been used in combination for the construction of whole cell biocatalysts displaying functional enzymes which were used as technological tools for the regio- and enantioselective synthesis of products, especially from substrates with several identical reactive groups, including sugars polyalcohols and steroids with high efficiency [12,21,22].

EP 02718168 describes autodisplay of adrenodoxin on an E. coli cell. Adrenodoxin belongs to the [2Fe-2S] ferredoxins, a family of small acidic iron-sulfur proteins. When displayed on the surface, the adrenodoxin is present in a non-functional form, because no prosthetic group is present. According to EP 02718168, a functional adrenodoxin attached to the cell surface can be obtained by contacting the adrenodoxin molecule with an exogenous [2Fe-2S] cluster serving as a prosthetic group.

The problem of the present invention is the provision of surface displayed enzymes comprising metal porphyrin-containing or flavin containing prosthetic groups. It was surprisingly found that by recombinant expression of these enzymes by surface display on a Gram-negative bacterium, a functional enzyme comprising the metal porphyrin-containing or flavin containing prosthetic group could be identified on the cell surface without introducing an exogenous prosthetic group, as described for enzymes containing [2Fe-2S] clusters in EP 02718168. In other words, polypeptides comprising prosthetic groups containing a metal porphyrin or a flavin can translocate to the cell surface in a conformation capable of retaining the prosthetic group when crossing the outer membrane, for example by mediation of the omp85 pathway. The prosthetic group may also be transported to the cell surface independently from the surface-displayed enzyme. Mechanisms are known for elimination of superfluous or toxic compounds, including compounds suitable as prosthetic groups (e.g., porphyrins), from the cell. In the present invention, it has been surprisingly found that metal porphyrins transported across the cell membrane into the extracellular space independently from the enzyme/autotransporter construct can contact the enzyme displayed on the cell surface to a form an active enzyme (see Example 2).

Thus, a first aspect of the present invention is a method for displaying a recombinant polypeptide containing a prosthetic group on the surface of a host cell, wherein the prosthetic group comprises a metal porphyrin or a flavin, said method comprising the steps:
(a) providing a host cell transformed with a nucleic acid fusion operatively linked with an expression control sequence said nucleic acid fusion comprising:
   (i) a portion encoding a signal peptide,
   (ii) a portion encoding the recombinant polypeptide to be displayed,
   (iii) optionally a portion encoding a protease recognition site,
   (iv) a portion encoding a transmembrane linker, and
   (v) a portion encoding the transporter domain of an autotransporter,
   and
(b) culturing the host cell under conditions wherein the nucleic acid fusion is expressed and the expression product comprising the recombinant polypeptide containing the prosthetic group is displayed on the surface of the host cell.

By the method of the present invention, a functional recombinant polypeptide can be displayed. As indicated above, display of the functional recombinant polypeptide of the present invention comprising a prosthetic group containing a metal porphyrin or a flavin does not require an exogenously added prosthetic group. In the present invention, the prosthetic group can be produced by the host cell (“endogenously produced prosthetic group”). In a preferred embodiment, the method of the present invention, in particular step (b), is performed with the proviso that the surface-displayed recombinant polypeptide is not contacted with an exogenous prosthetic group being a metal porphyrin or a flavin. In this context, “exogenous prosthetic group” refers to a prosthetic group not produced by the host cell.

The recombinant polypeptide to be displayed may also be termed “passenger”, “passenger polypeptide” or “passenger protein”.

Step (a) of the methods of the present invention refers to the provision of a host cell. The host cell used in the method of the present invention is preferably a bacterium, more preferably a Gram-negative bacterium, particularly an enterobacterium such as E. coli.

According to the present invention, a host cell, particularly a host bacterium is provided which is transformed with a nucleic acid fusion operatively linked with an expression control sequence, i.e. a promoter, and optionally further sequences required for gene expression in the respective host cell. The skilled person knows suitable promoters and expression control sequences. The promoter or/and the expression control sequence may be homologous or heterologous to the host cell. Preferably, the nucleic acid fusion is located on a recombinant vector, e.g. a plasmid vector. The host cell may be transformed with at least one nucleic acid fusion, for instance two, three, four, five or even more nucleic acid fusions. If two or more nucleic acid fusions are transformed into a host cell, the nucleic acid fusions preferably encode different recombinant polypeptides as described herein. If a host cell transformed with several nucleic acid fusions is used, these nucleic acid fusions may be located on a single vector or on a plurality of vectors.

At least one host cell as described herein, for instance two, three, four, five, six or even more host cells as described herein may be provided in the methods of the present invention. Each of these host cells is transformed with one nucleic acid fusion or at least one nucleic acid fusion, as described herein. Preferably, the nucleic acid fusions transformed in the at least one host cell encode different recombinant polypeptides as described herein.

The different recombinant polypeptides which may be provided in one or at least one host cell may form a functional unit, for instance the subunits of a functional unit, such as the subunits of an enzyme or the subunits or/and components of an enzyme complex.

The nucleic acid fusion comprises (i) a portion encoding a signal peptide, preferably a portion coding for a Gram-negative signal peptide allowing for transport into the periplasm through the inner cell membrane. The signal peptide may be a signal peptide homologous to the host cell. The signal peptide may also be a signal peptide heterologous to the host cell.

Further, the nucleic acid fusion comprises (ii) a portion encoding the recombinant polypeptide to be displayed.

Further, the nucleic acid fusion optionally comprises a portion encoding a protease recognition site, which may be a recognition site for an intrinsic protease, i.e. a protease naturally occurring in the host cell, or an externally added protease. For example, the externally added protease may be an IgA protease (cf. EP-A-0 254 090), thrombin or factor X. The intrinsic protease may be e.g. selected from OmpT, OmpK or protease X.

Furthermore, the nucleic acid fusion comprises (iv) a portion encoding a transmembrane linker which is required for the presentation of the passenger polypeptide (ii) on the outer surface of the outer membrane of the host cell. A transmembrane linker domain may be used which is homologous with regard to the autotransporter, i.e. the transmembrane linker domain is encoded by a nucleic acid portion directly 5′ to the autotransporter domain. Also a transmembrane linker domain may be used which is heterologous with regard to the autotransporter. The length of the transmembrane linker is preferably 30-160 amino acids.

Further, the nucleic acid fusion comprises (v) a transporter domain of an autotransporter. In the context of the present invention, autodisplay may be the recombinant surface display of proteins or polypeptides by means of an autotransporter in any Gram-negative bacterium. The transporter domain of the autotransporter according to the invention can be any transporter domain of an autotransporter and is preferably capable of forming a β-barrel structure. A detailed description of the β-barrel structure and preferred examples of β-barrel autotransporters are disclosed in WO97/35022 incorporated herein by reference. Henderson et al. (2004) describes autotransporter proteins which comprise suitable autotransporter domains (for summary, see Table 1 of Henderson et al., 2004). The disclosure of Henderson et al. (2004) is included herein by reference. For example, the transporter domain of the autotransporter may be selected from Ssp (P09489, S. marcescens), Ssp-h1 (BAA33455, S. marcescens), Ssp-h2 (BAA11383, S. marcescens), PspA (BAA36466, P. fluorescens), PspB (BAA36467, P. fluorescens), Ssa1 (AAA80490, P. haemolytica), SphB1 (CAC44081, B. pertussis), AspA/NalP (AAN71715, N. meningitidis), VacA (Q48247, H. pylori), AIDA-I (Q03155, E. coli), IcsA (AAA26547, S. flexneri), MisL (AAD16954, S. enterica), TibA (AAD41751, E. coli), Ag43 (P39180, E. coli), ShdA (AAD25110, S. enterica), AutA (CAB89117, N. meningitidis), Tsh (I54632, E. coli), SepA (CAC05786, S. flexneri), EspC (AAC44731, E. coli), EspP (CAA66144, E. coli), Pet (AAC26634, E. coli), Pic (AAD23953, E. coli), SigA (AAF67320, S. flexneri), Sat (AAG30168, E. coli), Vat (AAO21903, E. coli), EpeA (AAL18821, E. coli), EatA (AAO17297, E. coli), EspI (CAC39286, E. coli), EaaA (AAF63237, E. coli), EaaC (AAF63038, E. coli), Pertactin (P14283, B. pertussis), BrkA (AAA51646, B. pertussis), Tef (AAQ82668, B. pertussis), Vag8 (AAC31247, B. pertussis), PmpD (O84818, C. trachomatis), Pmp20 (Q9Z812, C. pneumoniae), Pmp21 (Q9Z6U5, C. pneumoniae), IgA1 protease (NP_{—}283693, N. meningitidis), App (CAC14670, N. meningitidis), IgA1 protease (P45386, H. influenzae), Hap (P45387, H. influenzae), rOmpA (P15921, R. rickettsii), rOmpB (Q53047, R. rickettsii), ApeE (AAC38796, S. enterica), EstA (AAB61674, P. aeruginosa), Lip-1 (P40601, X. luminescens), McaP (AAP97134, M. catarrhalis), BabA (AAC38081, H. pylori), SabA (AAD06240, H. pylori), AlpA (CAB05386, H. pylori), Aae (AAP21063, A. actinomycetemcomitans), NanB (AAG35309, P. haemolytica), and variants of these autotransporters. Given in brackets for each of the exemplary autotransporter proteins are examples of suitable genbank accession numbers and species from which the autotransporter may be obtained. Preferably the transporter domain of the autotransporter is the E. coli AIDA-I protein or a variant thereof, such as e.g. described by Niewert U., Frey A., Voss T., Le Bouguen C., Baljer G., Franke S., Schmidt M A. The AIDA Autotransporter System is Associated with F18 and Stx2e in Escherichia coli Isolates from Pigs Diagnosed with Edema Disease and Postweaning Diarrhea. Clin. Diagn. Lab. Immunol. 2001 Jan, 8(1):143-149; 9.

Variants of the above indicated autotransporter sequences can e.g. be obtained by altering the amino acid sequence in the loop structures of the β-barrel not participating in the transmembrane portions. Optionally, the nucleic acid portions coding for the surface loops can be deleted completely. Also within the amphipathic β-sheet conserved amino exchanges, i.e. the exchange of an hydrophilic by another hydrophilic amino acid or/and the exchange of a hydrophobic by another hydrophobic amino acid may take place. Preferably, a variant has a sequence identity of at least 70%, at least 90%, at least 95% or at least 98% on the amino acid level to the respective native sequence of the autotransporter domain, in particular in the range of the β-sheets:

Step (b) of the methods of the present invention refers to culturing the host cell under conditions wherein the nucleic acid fusion is expressed and the expression product comprising the recombinant polypeptide is displayed on the surface of the host cell. The person skilled in the art knows suitable culture conditions. The method according to the invention allows for an efficient expression of passenger proteins on the surface of host cells, particularly E. coli or other Gram-negative bacterial cells up to 100 000 or more molecules per cell by using a liquid medium of the following composition: 5 g/l to 20 g/l, preferably about 10 g/l trypton, 2 g/l to 10 g/l, preferably about 5 g/l yeast extract, 5 g/l to 20 g/l, in particular about 10 g/l NaCl and the remaining part water. The medium should possibly contain as little as possible divalent cations, thus preferably Aqua bidest or highly purified water, e.g. Millipore water is used. The liquid medium may contain in addition preferably EDTA in a concentration of 2 μM to 20 μM, in particular 10 μM. Moreover, it contains preferably reducing reagents, such as 2-mercapto ethanol or dithiotreitol or dithioerythritol in a preferred concentration of 2 mM to 20 mM. The reducing reagents favour a non-folded structure of the polypeptide during transport. The liquid medium can further contain additional C-sources, preferably glucose, e.g. in an amount of up to 10 g/l, in order to favour secretion i.e. transfer of the passenger to the surrounding medium. For surface display preferably no additional C-source is added. Preferred culture conditions for Gram-negative cells, such as E. coli, are described in the Examples.

If the host cell is a Gram-negative bacterium, the polypeptide synthesized in the cytoplasma can be translocated from the cytoplasm into the periplasmic space by crossing the inner membrane. This can be effected by the signal peptide.

While not wishing to be bound by theory, display of a functional polypeptide comprising a metal porphyrin or a flavin on the surface of a Gram-negative cell by autodisplay can be explained as follows. In a first step the prosthetic group comprising a metal porphyrin or a flavin is introduced into the polypeptide of the present invention in the periplasmic space. In a second step, the recombinant polypeptide of the present invention is translocated from the periplasmic space onto the cell surface in a conformation capable of retaining the prosthetic group when crossing the outer membrane, for example via the omp85 pathway. By this procedure, a functional polypeptide attached to the cell surface can be obtained. In a different mechanism, the prosthetic group, present in the periplasmic space, may be transported independently from the recombinant polypeptide across the outer membrane. A suitable transporter is the outer membrane channel-tunnel protein TolC, in particular for the transportation of metal porphyrins. Both mechanisms may account for transportation of at least a part of prosthetic group transported to the cell surface.

In the present invention, the prosthetic group can be transported to the cell surface by any suitable transport protein, which may be recombinantly expressed in the host cell. This transport can be independent from the autotransporter. The prosthetic group being a metal porphyrin can preferably be transported to the cell surface by a TolC-dependent mechanism. The prosthetic group being a metal porphyrin can also be transported to the cell surface by TolC or/and another suitable transport protein. In Gram negative cells, the prosthetic group being a metal porphyrin can preferably be transported across the outer membrane surface by TolC.

In the present invention, any TolC polypeptide may be employed. For example, an E. coli TolC may be employed.

In particular, the TolC polypeptide is homologous to the host cell. For example, an E. coli TolC may be employed in an E. coli host cell.

The TolC polypeptide may be a recombinant TolC. For example, the TolC polypeptide may be recombinantly expressed in the host cell. TolC may be over-expressed in the host cell. If, for example, the host cell has only low expression of TolC and thus only low capability of porphyrin transport to the cell surface, TolC may be over-expressed.

The TolC polypeptide, as used herein, may comprise a sequence selected from
(a) SEQ ID NO:8, and
(b) sequences having at least 70%, at least 80%, at least 90%, at least 95%, or at least 98% identity to the sequence of (a).

The TolC polypeptide, as used herein, may be encoded by a sequence selected from
(a) nucleic acid sequences encoding the amino acid sequences of SEQ ID NO:8,
(b) nucleic acid sequences encoding amino acid sequences having at least 70%, at least 80%, at least 90%, at least 95%, or at least 98% identity to the amino acid sequence of SEQ ID NO:8,
(c) SEQ ID NO:7, and
(d) sequences having at least 70%, at least 80%, at least 90%, at least 95%, or at least 98% identity to the sequence of (c).

In particular, nucleic acid sequences of (a), (b) and (d) include sequences within the scope of the degeneracy of the genetic code.

The TolC polypeptide, as defined herein, may be a HasF polypeptide, for example from Serratia marcescens.

If the passenger polypeptide is transported together with the prosthetic group to the cell surface, the passenger may acquire the prosthetic group within the cell. In the method of the present invention, in step (b), the prosthetic group endogenously produced in the cell may be introduced into the polypeptide of the present invention within the periplasmic space.

In the method of the present invention, step (b) may involve the omp85 pathway. Step (b) may comprise transportation of the polypeptide of the present invention via the omp85 pathway. Step (b) may comprise translocation of the polypeptide of the present invention from the periplasmic space onto the cell surface by the omp85 pathway, in particular in a conformation capable of retaining the prosthetic group when crossing the outer membrane.

In the present invention, any Omp85 or Omp85 homologue may be employed. “Omp85”, as used herein, includes homologues of Omp85. For example, the Omp85 homologue YaeT from E. coli may be employed. The Omp85, in particular YaeT, may comprise a sequence selected from
(a) SEQ ID NO:3 and SEQ ID NO:4, and
(b) sequences having at least 70%, at least 80%, at least 90%, at least 95%, or at least 98% identity to the sequence of (a).

Also employed may be a nucleic acid encoding an Omp85. The nucleic acid encoding Omp85, in particular YaeT, may comprise a nucleic acid sequence selected from
(a) nucleic acid sequences encoding the amino acid sequence of SEQ ID NO:3 and SEQ ID NO:4, and
(b) nucleic acid sequences encoding amino acid sequences having at least 70%, at least 80%, at least 90%, at least 95%, or at least 98% identity to the amino acid sequence of SEQ ID NO:3 or/and SEQ ID NO:4,

In particular, nucleic acid sequences of (a) and (b) include sequences within the scope of the degeneracy of the genetic code.

The components (i) to (v) in the nucleic acid fusion of the present invention are preferably oriented from 5′ to 3′. In the expression product obtained in step (b), the amino acid sequences encoded by nucleic acid sequences (i) to (v) are preferably arranged N terminal to C terminal.

The method of the present invention may comprise preparing a membrane preparation from the cell obtained in step (b). The membrane preparation may comprise membrane particles. The membrane particles may be membrane vesicles. Preferred membrane particles are outer membrane particles. In particular the method of the present invention may comprise preparing outer membrane particles of cells displaying a recombinant polypeptide on the surface, e.g. of Gram-negative bacterial cells. The person skilled in the art knows suitable conditions (e.g. Hantke, 1981, Schultheiss et al., 2002). Typical conditions for preparing membrane particles are employed in the examples of the present invention. Outer membrane particles from a host cell as described herein may be performed by a method comprising the steps:
(a) treating the host cell with a hydrolase (such as lysozyme) and optionally with a DNAse. This enzymatic treatment may be performed at room temperature. The hydrolase hydrolyses the cell wall within the periplasmic space. The cell wall comprises peptidoglycans to be hydrolyzed.
(b) optionally solubilizing the preparation of (a) with a tenside, such as Triton X-100, or/and with sarcosine, followed by optional centrifugation of cell debris. The thus obtained preparation of outer membrane particles may be centrifuged, washed and resuspended.

The diameter of the membrane particles may be in the range of 1 nm to 1000 nm, in the range of 50 nm to 500 nm, in the range of 75 to 200 nm, or in the range of 90 to 120 nm. At least 80%, at least 90%, at least 95%, or at least 98% of the membrane particles may have a diameter in a range selected from the ranges described herein.

In a host cell being a Gram-negative bacterium, such as E. coli, after translocation, the recombinant passenger remains attached to the surface of the outer membrane by the β-barrel, which is serving as an anchor within the outer membrane. Due to the controlled integration of the β-barrel within the outer membrane, the C terminal part of the β-barrel is directed to the inner side of the outer membrane, whereas the N-terminal part of the linker, to which the recombinant passenger protein is covalently bound, is directed to the outer surface of the outer membrane, i.e. the environment. The recombinant passenger protein has an oriented location after transport, namely it is directed to the cellular surface. The recombinant passenger protein has the identical orientation as the lipopolysaccharide (LPS) layer which may be present in the outer membrane.

Membrane particles of the present invention prepared from the host cell of the present invention comprise the recombinant peptide at the surface directed to the environment. In contrast to the inner membrane which is a unit membrane, the outer membrane of Gram-negative bacteria, in particular E. coli, is asymmetric. The outer membrane may comprise an inner layer comprising phospholipids and an outer layer comprising LPS. LPS is hydrophilic and may contain several negative charges. By using outer membrane particles with anchored passenger proteins by a β-barrel for the coating of carriers, the outer side of the outer membrane, in particular the LPS side will be directed to the surface distal to the carrier. As a consequence the recombinant protein or a domain thereof, which are integrated in the outer membrane by autodisplay, will be directed to the surface distal to the carrier as well. The core part of the membrane particles may stabilize the interaction of the outer membrane layer obtained by applying outer membrane particles to the carrier by hydrophobic interactions and may contain lipoproteins or peptidoglycans.

A preferred prosthetic group is a metal porphyrin, as described herein.

The prosthetic group being the metal porphyrin may comprise a heavy metal such as cobalt, nickel, manganese, copper and iron. The metal porphyrin of the present invention in particular comprises a heme group.

Another preferred prosthetic group is a flavin, as described herein.

The prosthetic group being the flavin may be selected from FAD and FMN.

The polypeptide of the present invention comprising a prosthetic group preferable is an enzyme.

The polypeptide comprising the metal porphyrin may be an enzyme. The polypeptide comprising the metal porphyrin may be selected from P450 enzymes (such as P450 reductases) and cytochromes (such as cytochrome b5). The polypeptide comprising the metal porphyrin may be selected from hemoproteins. In particular, the polypeptide comprising the metal porphyrin may be selected from monooxygenases. The polypeptide comprising the metal porphyrin may be selected from CYP11B1, CYP11A1, CYP106A2 and CYP3A4. The polypeptide comprising the metal porphyrin may preferably be selected from CYP106A2 and CYP3A4.

In a preferred embodiment, the polypeptide comprising the metal porphyrin comprises a sequence selected from
(a) SEQ ID NO:2 and SEQ ID NO:6, and
(b) sequences having at least 70%, at least 80%, at least 90%, at least 95%, or at least 98% identity to the sequence of (a).

The portion (ii) of the nucleic acid fusion of the present invention may encode a polypeptide which, when functional, comprises the metal porphyrin, as described herein. The portion (ii) of the nucleic acid fusion of the present invention may comprise a nucleic acid sequence selected from
(a) nucleic acid sequences encoding the amino acid sequences of SEQ ID NO:2 and SEQ ID NO:6,
(b) nucleic acid sequences encoding amino acid sequences having at least 70%, at least 80%, at least 90%, at least 95%, or at least 98% identity to the amino acid sequence of SEQ ID NO:2 or/and SEQ ID NO:6,
(c) SEQ ID NO:1 and SEQ ID NO:5, and
(d) sequences having at least 70%, at least 80%, at least 90%, at least 95%, or at least 98% identity to the sequence of (c).

In particular, nucleic acid sequences of (a), (b) and (d) include sequences within the scope of the degeneracy of the genetic code.

The skilled person knows suitable methods to determine the degree of identity of nucleic acid sequences and amino acid sequences. Known algorithms, such as BLAST (for nucleic acids) or PBLAST (for amino acid sequences) may be used. A nucleic acid or polypeptide comprising sequences having at least 70%, at least 80%, at least 90%, at least 95%, or at least 98% identity to a given sequence includes fragments of the given nucleic acid or polypeptide.

The flavin containing polypeptide of the present invention may be selected from flavoproteins, in particular FAD or FMN containing proteins. Preferred are FAD containing proteins. The flavin containing polypeptide may be selected from enzymes such as oxidoreductases, NADH oxidases, dehydrogenases, and oxidases, especially sugar oxidases, such as pyranose oxidase. The NADH oxidase is in particular an FAD containing enzyme.

The polypeptide of the present invention to be displayed on the surface of the cell may be a multimeric polypeptide. The multimeric recombinant polypeptide may be a homodimer, i.e. a polypeptide consisting of two identical subunits or a homomultimer, i.e. a polypeptide consisting of three or more identical subunits. On the other hand, the multimeric recombinant polypeptide may be a heterodimer, i.e. a polypeptide consisting of two different subunits or a heteromultimer consisting of three or more subunits wherein at least two of these subunits are different. For example, the multimeric polypeptide is comprised of a plurality of subunits which form a “single” multimeric polypeptide or a complex of a plurality of functionally associated polypeptides which may in turn be monomeric and/or multimeric polypeptides. It should be noted that at least one subunit of the multimeric recombinant protein may contain at least one prosthetic group as described herein. Further, is should be noted that the nucleic acid fusion may encode a plurality of polypeptide subunits as a polypeptide fusion which when presented on the cell surface forms a functional multimeric polypeptide.

Homodimers or homomultimers may be formed by a spontaneous association of several identical polypeptide subunits displayed on the host cell membrane. Heterodimers or heteromultimers may be formed by a spontaneous association of several different polypeptide subunits displayed on the host cell membrane.

On the other hand, a multimeric recombinant polypeptide may be formed by an association of at least one polypeptide subunit displayed on the host cell membrane, as described herein, and at least one soluble polypeptide subunit added to the host cell membrane. The added subunit may be identical to the displayed subunit or be different therefrom.

Yet another aspect of the present invention is a host cell displaying the recombinant polypeptide on the surface. The host cell may be any host cell as described herein, in particular a host cell displaying a recombinant polypeptide on the surface thereof, wherein the recombinant polypeptide contains a prosthetic group comprising a metal porphyrin or a flavin, and wherein the recombinant polypeptide comprises
(I) a portion comprising the recombinant polypeptide to be displayed,
(II) optionally a portion comprising a protease recognition site,
(III) a portion comprising a transmembrane linker, and
(IV) a portion comprising the transporter domain of an autotransporter.

The displayed polypeptide is in particular a functional polypeptide.

The portions (I) to (IV) of the recombinant polypeptide displayed by the host cell of the present invention are encoded in particular by the components (ii), (iii), (iv) and (v) of the nucleic fusion, as described herein.

Yet another aspect of the present invention is a membrane preparation comprising a recombinant polypeptide. The membrane preparation of the present invention may comprise membrane particles, as described herein. The membrane preparation may be obtained from a host cell as described herein. The recombinant polypeptide of the may be any recombinant polypeptide as described herein.

Yet another aspect of the present invention is the use of a membrane preparation comprising a recombinant polypeptide in the manufacture of a carrier comprising a recombinant polypeptide.

The membrane preparation of the present invention may be employed for coating a carrier. The carrier may comprise a membrane preparation of the present invention, as described herein.

The carrier may comprise a hydrophobic surface. The hydrophobic surface may have a contact angle of more than 90°. A increasing surface angle of more than 30° indicates a gradually increasing hydrophobicity of a surface. In the present context, a hydrophobic surface may have a contact angle of at least 40°. The surface preferably has a hydrophobicity described by a contact angle of at least 40°, at least 50°, at least 60°, at least 65°, at least 70°. Contact angles are preferably determined by the sessile drop method. The sessile drop method is a standard method for determining contact angles. Measurements may be performed with a contact angle goniometer. Preferred contact angles of the hydrophobic surface are in a range of 40° to 100°, 50° to 90°, or 60° to 80°.

The surface of the carrier may be a metal surface. A suitable metal surface has a contact angle e.g. in the range of 50° to 80°. A suitable metal may be selected from gold, silver, titanium, aluminium and alloys such as brass. A preferred surface is a gold surface. The gold surface may be employed as it is. An untreated gold surface has a hydrophobicity suitable for the carrier as described herein. A treatment of the gold surface with thiolated hydrocarbons or hydrocarbons with functional groups such as carboxylic acids or hydroxyl groups is not required.

Another preferred surface of the carrier comprises a polymer, for instance a surface usually employed in disposable materials for use in biochemical or/and medical science. The polymer may be an artificial polymer. Examples of artificial polymers include a polymer selected from polystyrenes, polypropylenes, and polycarbonates. The polystyrene may be produced from [2,2]paracyclophane monomers. Polystyrene surfaces may be treated with oxygene plasma introducing OH or/and methylene groups in order to modify the hydrophobicity. Examples of such modified surfaces include Maxi-sorp, Medi-sorp, Multi-sorp, and Poly-sorp surfaces. Another suitable polystyrene surface is Parylene N produced from [2,2]paracyclophane monomers. Yet another suitable surface is Parylene A [Poly(monoamino-p-xylene)]. Especially suitable are surfaces comprising a polymer having a hydrophobicity described by a contact angle of at least 50°. Suitable surfaces are selected from polystyrene, Parylene A, Parylene N, Maxi-sorp, Medi-sorp, Multi-sorp, and Poly-sorp. Preferred surfaces are selected from polystyrene, Parylene A, Parylene N, Maxi-sorp, Medi-sorp, and Poly-sorp.

The surface may comprise a natural polymer. Suitable natural polymers include polybutyrate and cellulose and derivatives thereof. A further surface is provided by latex particles, in particular latex beads.

Yet another surface is provided by C18-modified particles, in particular C18-modified monolithic silica particles. C18 refers to an alkyl group comprising 18 carbon atoms. C18-modified particles are known in the art.

Yet another suitable surface is a glass surface.

The surface may be modified is order to adjust the hydrophobicity. Modification may be performed by chemical treatment (i.e. by oxygen plasma), physical treatment (e.g. by laser irradiation or/and radioactive irradiation), or by mechanical treatment.

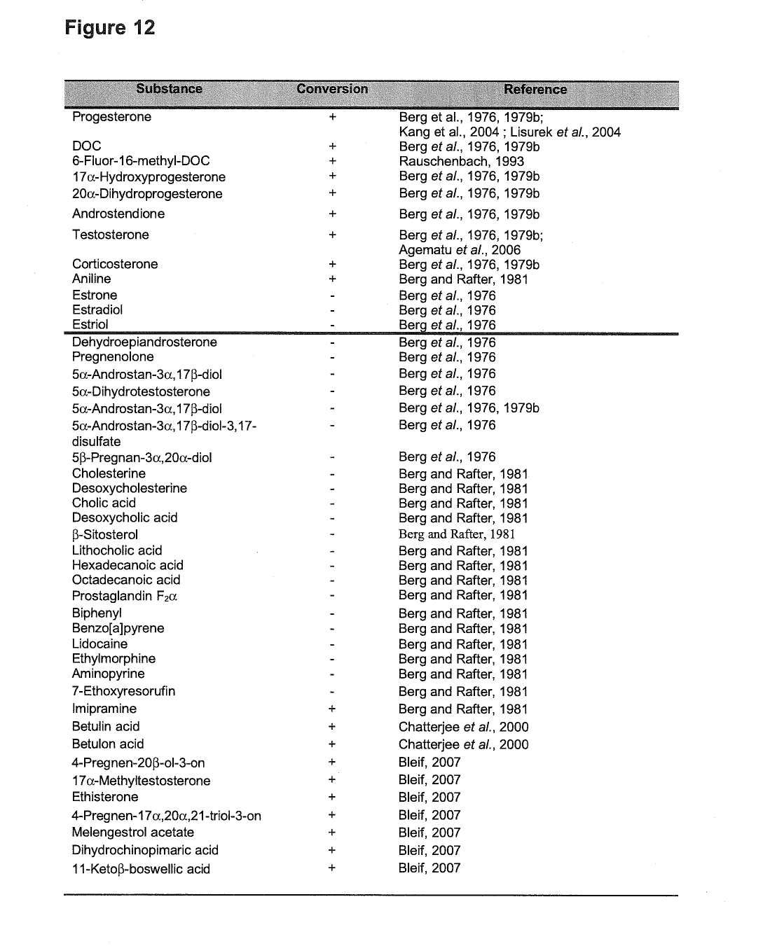

The method according to the invention and the host cells according to the invention can be used for a variety of different applications, e.g. as whole cell biofactories or membrane preparation biofactories for chemical synthesis procedures, e.g. for the synthesis of organic substances selected from enzyme substrates, drugs, hormones, starting materials and intermediates for syntheses procedures and chiral substances (cf. Roberts, Chemistry and Biology 6 (1999), R269-R272). Typical CYP106A2 substrates are described in FIG. 12.

In particular, the method according to the invention and the host cells according to the invention, as described herein, can be used in the chemical synthesis, for example in enzymatically catalyzed enantioselective or/and regioselective steps. For example, CYP106A2 displayed on the surface of a cell, as described herein, can be used for the conversion of steroids, for the conversion of abietic acid, or for the preparation of desipramine from imipramine, as exemplified by Examples 1 and 3.

Furthermore, the cell or the membrane preparation of the invention may be used for a directed evolution procedure, e.g. for the development of new biocatalysts for the application in organic syntheses.

This is achieved in a particular embodiment by varying the amino acid sequence of the polypeptide containing a prosthetic group selected from metal porphyrins and flavins, as described herein, via site-specific or random mutagenesis and by testing variant carrying cells or membrane preparations or libraries containing variant carrying cells or membrane preparations thereof using a certain chemical reaction with the help of suitable screening methods, in particular high throughput screening (HTS) methods for the conversion of a certain substrate.

In yet another preferred embodiments libraries of variants of a polypeptide containing a prosthetic group selected from metal porphyrins and flavins, as described herein, are examined in view of the role of defined amino acids during certain functions, in particular catalytic functions.

In general, these particular embodiments concern the production of variants of proteins and/or enzymes and the production of libraries with variants of proteins and/or enyzmes, respectively, which carry a prosthetic group, as described herein, or multimers etc. and which are screened in view of a certain characteristic, i.e. one or optionally several variants fulfilling this desired characteristic perfectly are selected. By selecting the variant the cell is selected, too, and carries the nucleic acid coding the variant. Thus, at the same time both the amino acid sequence and the structural information of the variant can be determined via the nucleic acid sequence. The characteristics in question are particularly enzyme inhibiting, catalytical, toxin degrading, synthesizing, therapeutical etc. characteristics.

Moreover, the host cell or the membrane preparation may be used as an assay system for a screening procedure, e.g. for identifying modulators (activators or inhibitors) of displayed polypeptides, containing a prosthetic group selected from metal porphyrins and flavins, as described herein, which may be used as potential therapeutic agents. The screening procedure may also be used to identify variants of displayed polypeptides having predetermined desired characteristics. For this purpose, libraries of modulators and/or libraries of displayed polypeptides may be used. Further, the host cells or membrane preparations derived therefrom may be used as a system for toxicity monitoring and/or degrading toxic substances in the environment, in the laboratory or in biological, e.g. human, animal, or non-biological systems.

An essential advantage of applying the host cells and membranes according to the invention is enabling correct folding and biological activity of proteins or protein complexes, e.g. of the polypeptide containing a prosthetic group selected from metal porphyrins and flavins, as described herein, which require a membrane environment. Thus, a reconstitution as previously considered to be necessary is no longer required. Thereby the production steps of a functional biocatalytic system are simplified and an increased stability of the system per se is obtained.

Further preferred examples for the recombinant polypeptide to be displayed, i.e. the passenger polypeptides are peptides or proteins selected from the group of drug metabolizing enzymes, such as CYP1A2 involved in the activation of aromatic amine carcinogenes, heterocyclic arylamine promutagenes derived from food pyrolysates and aflatoxin B1 (Gallagher E P, Wienkers L C, Stapleton P L, Kunze K L, Eaton D L., Role of human microsomal and human complementary DNA-expressed cytochromes P4501A2 and P4503A4 in the bioactivation of aflatoxin B1. Cancer Res. 1994, Jan 1; 54(1):101-8) or CYP2E1 capable of activating the procarcinogenes N-nitrosodimethylamine and N-nitrosodiethylamin and metabolizes the procarcinogenes benzene, styrene, carbon tetrachloride, chloroform (Yoo J S, Ishazaki H, Yang C S., Roles of cytochrome P450IIE1 in the dealkylation and denitrosation of N-nitrosodimethylamine and N-nitrosodiethylamine in rat liver microsomes. Carcinogenesis. 1990 December; 11(12):2239-43; Peter R, Bocker R, Beaune P H, Iwaskai M, Guengerich F P, Yang C S., Hydroxylation of chlorzoxazone as a specific probe for human liver cytochrome P-450IIE1. Chem. Res. Toxicol. 1990 November-December; 3(6):566-73). Further preferred passenger peptides are peptides from the group of steroid biosynthesis enyzmes, such as CYP11B1 involved in the formation of cortisol and aldosterone (Bernhardt R., Cytochrome P450: structure, function and generation of reactive oxygen species. Rev. Physiol. Biochem. Pharmacol. 1996; 127:137-221) or CYP19 involved in the conversion of adrostenedione to 19-hydroxyandrostenedione, 19-oxo-androstenedione and estrone (Ryan K J., Biological aromatization of steroids. J. Biol. Chem. 1959; 134:268). Further preferred metal ion containing enzymes are Cu-containing enzymes, such as cytochrome-oxidase, Mn-containing enzymes, such as arginase and ribonucleotide reductase, Mo-containing enzymes, such as dinitrogenase and Se-containing enzymes, such as glutathione peroxidase.

Preferably the P450 enzymes are hepatic P450 enzymes, particularly P450 3A4, 2D6, 2C9 and 2C19. The host cells and/or preparations according to the invention are preferably used sequentially for testing the enzyme inhibition of P450 enzymes. For example, with the help of the host cell and/or membrane preparation according to the invention it can be found out in an early step of drug discovery, the so-called lead identification, whether the new drug lead structure to be tested could possibly have side-effects or lead to the so-called drug-drug interaction.

Further, the present invention shall be further illustrated by the following figures and examples:

FIG. 1: A: Schematic drawing of the hydroxylation reaction in the 15β-position of the steroid 11-deoxycorticosterone (DOC) catalyzed by CYP106A2. Redox equivalents are transferred from NADPH via the proteins AdR and Adx to the steroid converting enzyme CYP106A2. B: Oxidation of terfenadine by CYP3A4 to fexofenadine.

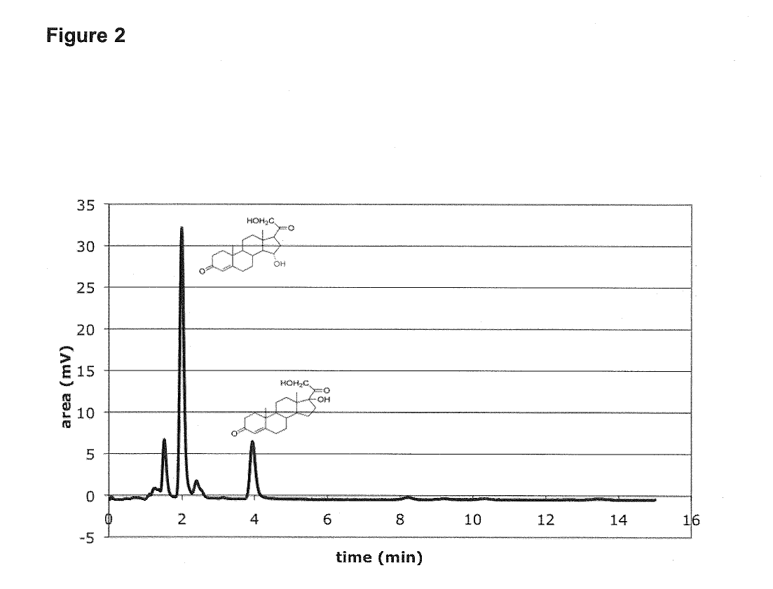

FIG. 2: Chromatogram of the CYP106A2 activity assay. Conversion of 11-deoxycorticosterone into 15beta-deoxycorticosterone using the pure enzyme. (Reichstein's Compound S, RSS, internal standard)

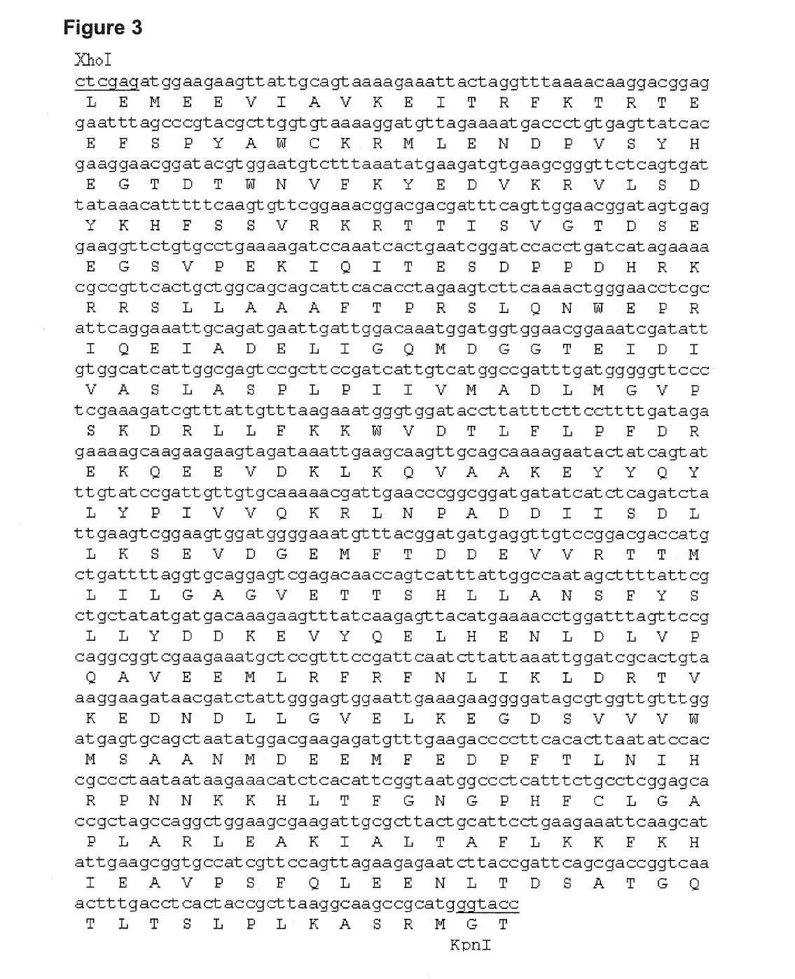

FIG. 3: Sequence of CYP106A2. Nucleic acid sequence (SEQ ID NO:1) and derived amino acid sequence (SEQ ID NO:2) of the CYP106A2 insert in plasmid pET-CYP13.

FIG. 4: A: Secretion mechanism of the autotransporter proteins in Gram-negative bacteria. B: Structure of a typical artificial autotransporter protein used in autodisplay (SEQ ID NO:9 and SEQ ID NO:10). C: Structure of the CYP106A2 fusion protein. Illustration of the fusion proteins necessary for the expression of CYP106A2. Important restriction sites for cloning are underlined (SEQ IDs NO:11-14).

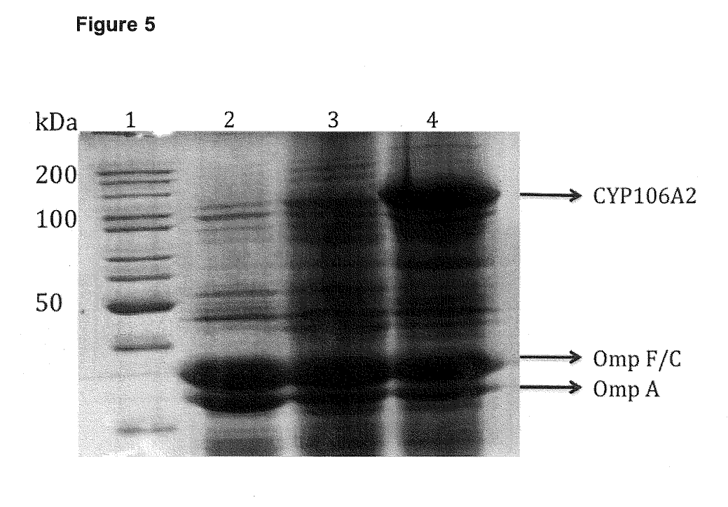

FIG. 5: Expression of CYP106A2. SDS-PAGE (10%) and Coomassie staining of outer membrane preparations obtained form E. coli BL21(DE3) pET-CYP13. 1: marker proteins, 2: control, BL21(DE3) without plasmid, 3: BL21 (DE3) pET-CYP13-IPTG, 4: BL21 (DE3) pET-CYP13+1 mM IPTG.

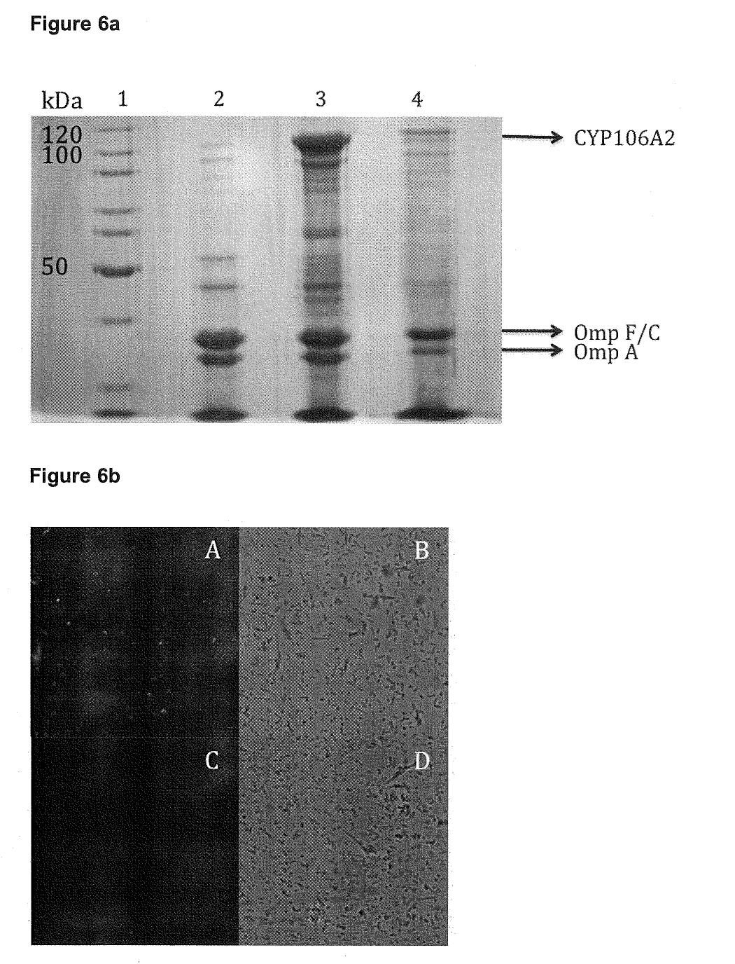

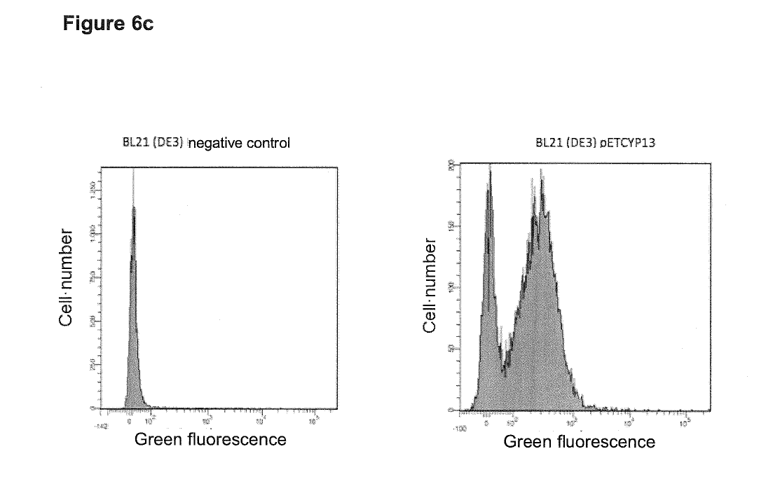

FIG. 6: a: Surface display of CYP106A2. Whole cell trypsin digestion and subsequent SDS-PAGE (10%) and Coomassie staining of outer membrane preparations obtained from E. coli BL21(DE3) pET-CYP13. 1: marker proteins, 2: control, BL21(DE3) without plasmid, 3: BL21 (DE3) pET-CYP13+IPTG−trypsin, 4: BL21 (DE3) pET-CYP13+1 IPTG+trypsin. b: Proof of successful surface display of CYP106A2 by indirect immune fluorescence A: E. coli BL21 (DE3) pETCYP13, abs.: 490 nm, em.: 520 nm, B: E. coli BL21 (DE3) pETCYP13, transmitted light, C: E. coli BL21 (DE3), abs.: 490 nm, em.: 520 nm, D: E. coli BL21 (DE3), transmitted light. All samples were prepared with two antibodies: a primary polyclonal anti-CYP106A2 antibody and a secondary FITC-labelled detection antibody. Only the cells containing the expression plasmid showed a positive reaction (see A). c: Proof of successful surface display of CYP106A2 by flow cytometry. All samples were prepared with two antibodies: a primary polyclonal anti-CYP106A2 antibody and a secondary FITC-labelled detection antibody. The mean fluorescence (mF) of the labelled cells was determined by FACS analysis; BL21(DE3) (negative control), mF=27; cells displaying CYP106A2 on the surface (BL21(DE3) pETCYP13), mF=268.

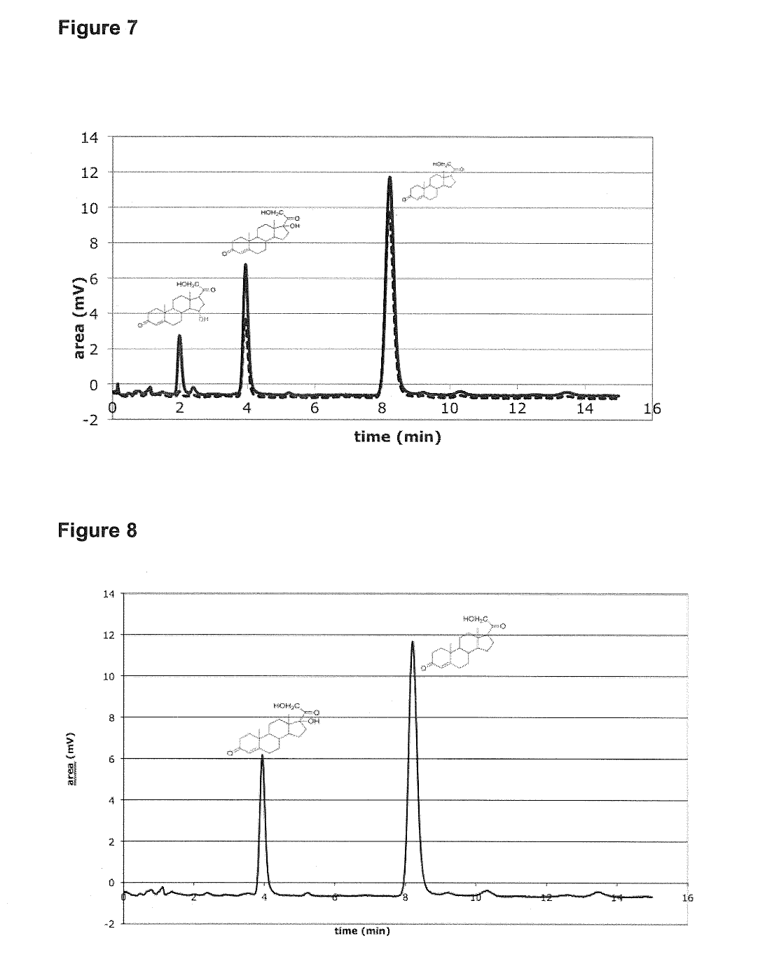

FIG. 7: Chromatogram of the activity assay with whole cells displaying CYP106A2. Conversion of 11-deoxycorticosterone into 15beta-deoxycorticosterone by BL21(DE3) pETCYP13. For that purpose cells were cultivated and half of them induced with 1 mM IPTG. Formation of the product only occurs after induced protein expression. (Reichstein's Compound S, RSS, internal standard)

[symbol] BL21(DE3) pET CYP 13 without addition of IPTG;

[symbol] BL21(DE3) pET CYP 13 with addition of 1 mM IPTG

FIG. 8: Chromatograms of the CYP106A2 activity assay using BL21(DE3) pETCYP13 cells without addition of Adx (additional negative control). Cells were cultivated and protein expression was induced with 1 mM IPTG. To proof that all conversions took only place on the surface of E. coli and not inside the cell by other electron supplying systems, substrate conversions were done without the addition of Adx, since it is too large of a molecule to enter the cell envelope. The chromatograms of this conversion assay shows, as expected, no product peak. (Reichstein's Compound S, RSS, internal standard).

[symbol] BL21(DE3) pET CYP 13 with addition of 1 mM IPTG

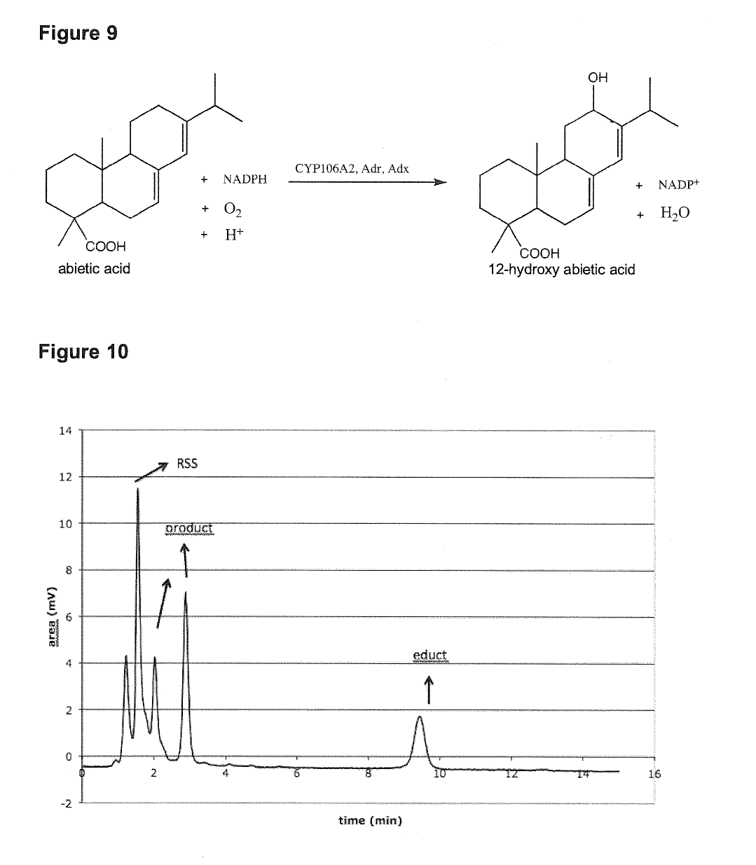

FIG. 9: Schematic drawing of the hydroxylation of abietic acid catalysed by CYP106A2. Redox equivalents are transferred from NADPH via the proteins AdR and Adx to CYP106A2.

FIG. 10: Chromatogram of the CYP106A2 activity assay using purified enzyme. Conversion of the non membrane transferrable educt abietic acid into the two products 12-alpha und 12-beta-Hydroxy-abietic acid by the purified enzyme CYP106A2. (Reichstein's Compound S, RSS, internal standard).

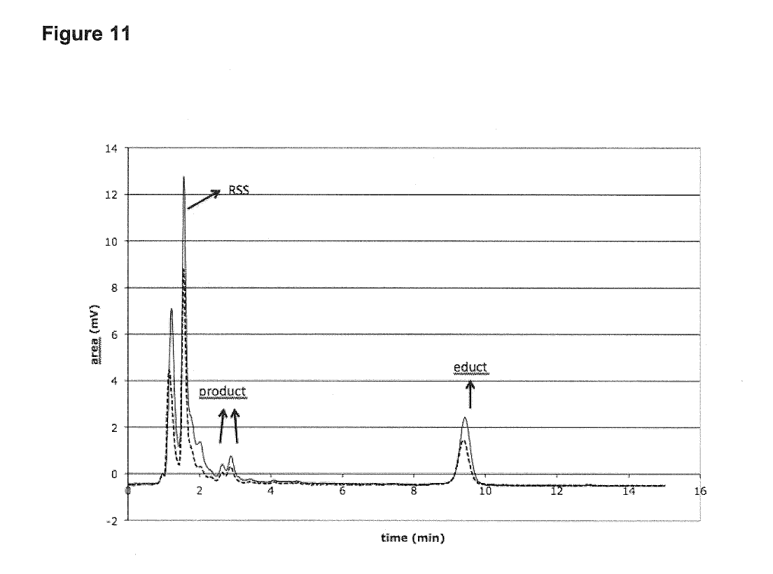

FIG. 11: Chromatogram of the activity assay using BL21(DE3) cells displaying CYP106A2. Conversion of the non membrane transferrable educt abietic acid into the two products 12-alpha and 12-beta-hydroxy-abietic acid by BL21(DE3) pETCYP13. For that purpose cells were cultivated and half of them induced with 1 mM IPTG. Formation of the product occurs in particular after induced protein expression. (Reichstein's Compound S, RSS, internal standard).

[symbol] BL21(DE3) pET CYP 13 without addition of IPTG;

[symbol] BL21 (DE3) pET CYP 13 with addition of 1 mM IPTG

FIG. 12: Table of known CYP106A2 substrates from Bacillus megaterium ATCC 13368.

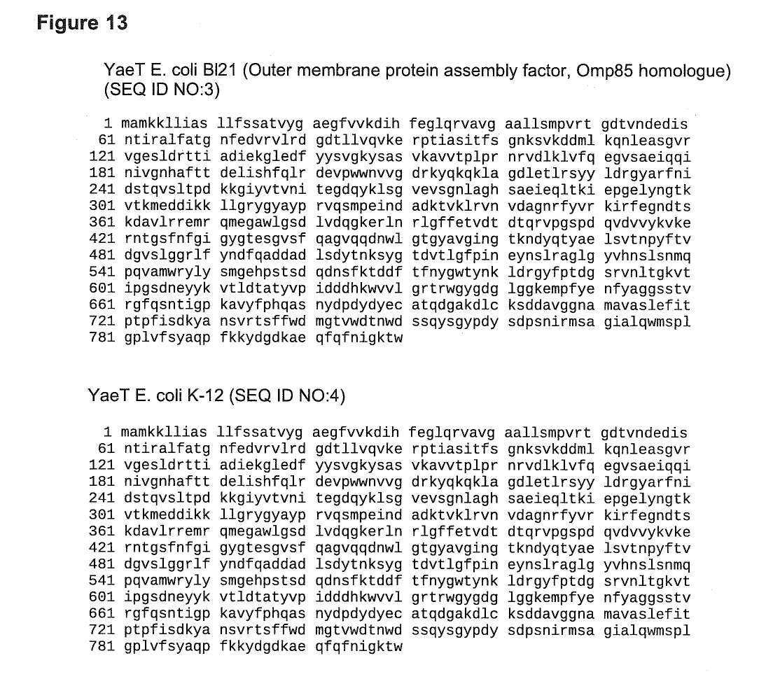

FIG. 13: Sequence of YaeT (Outer membrane protein assembly factor, Omp85 homologue) in E. coli strains B121 (SEQ ID NO:3) and K-12 (SEQ ID NO:4).

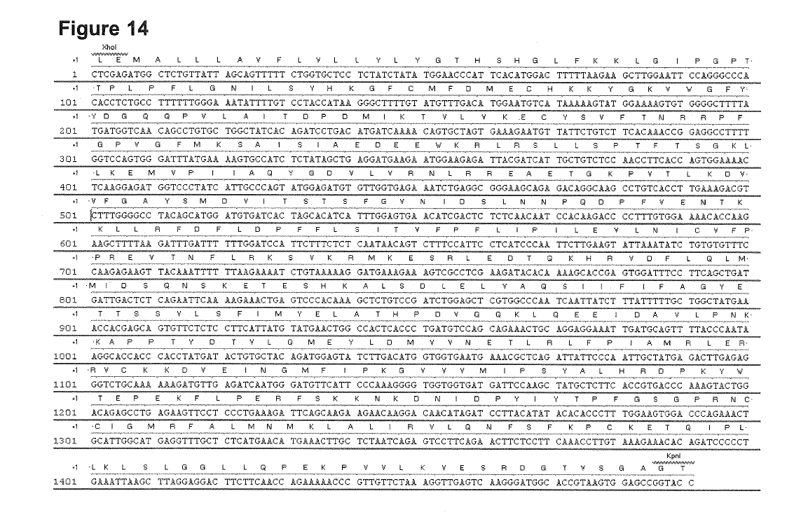

FIG. 14: Sequence of CYP3A4. Nucleic acid sequence (SEQ ID NO:5) and derived amino acid sequence (SEQ ID NO:6) of the CYP3A4 insert in plasmid pSC001 used for autodisplay of CYP3A4.

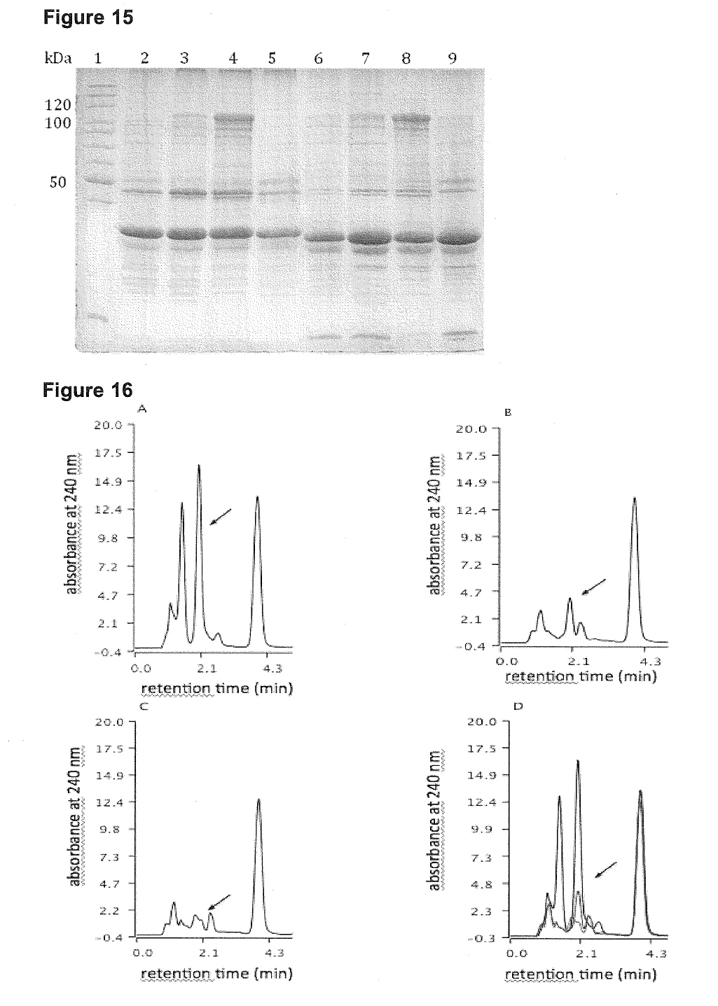

FIG. 15: Surface display of CYP106A2 in TolC negative cells. Whole cell proteinase k digestion and subsequent SDS-PAGE (12.5%) and Coomassie staining of outer membrane preparations obtained from E. coli BL21(DE3) pET-CYP13 and JW 5503 (DE3) pETCYP13. 1: marker proteins, 2: control, BL21(DE3) without plasmid, 3: BL21 (DE3) pET-CYP13−IPTG−proteinase k 4: BL21 (DE3) pET-CYP13+IPTG−proteinase k, 5: BL21 (DE3) pET-CYP13+1 IPTG+proteinase k, 6: control, JW 5503 (DE3) without plasmid, 7: JW 5503 (DE3) pET-CYP13−IPTG−proteinase k 8: JW 5503 (DE3) pET-CYP13+IPTG−proteinase k, 9: JW 5503 (DE3) pET-CYP13+1 IPTG+proteinase k.

FIG. 16: HPLC chromatograms showing CYP106A2 conversion of 11-deoxycorticosterone to 15β-deoxycorticosterone A: positive control (purified CYP106A2 enzyme). B: TolC positive cells (BL21 (DE3) pETCYP13) induced with IPTG. C: TolC negative cells (JW 5503 (DE3) pETCYP13) induced with IPTG. D: Overlay of the 3 graphs. The major product, 15β-deoxycorticosterone, was seen at a retention time of 2 min. The amount of this product decreased when CYP106A2 was expressed in the E. coli strain lacking the TolC channel protein. The peak at the retention time of 4 min is the internal standard Reichstein's compound S.

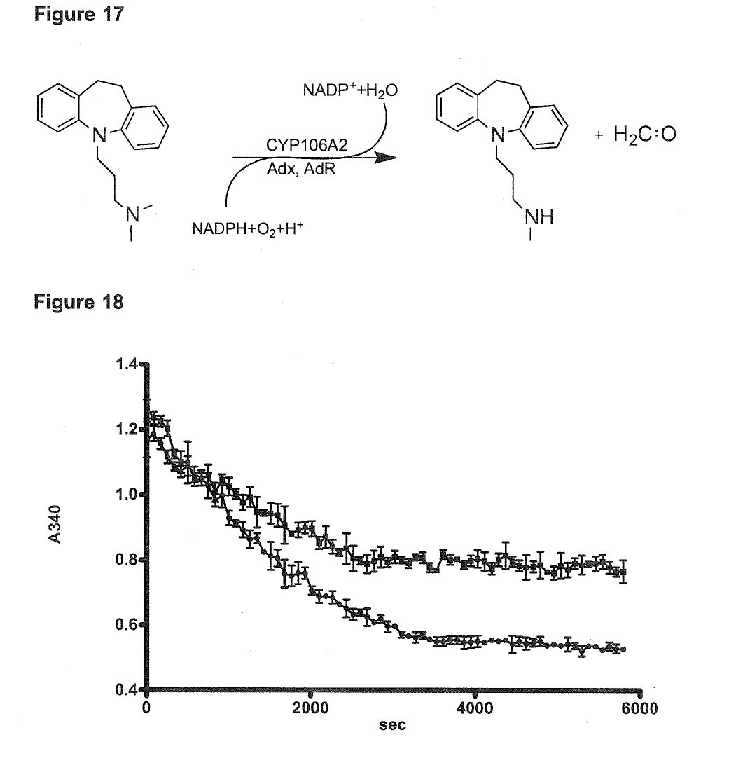

FIG. 17: Schematic drawing of the N-demethylation of the antidepressant imipramine into desipramin. The reaction is catalyzed by CYP106A2 displayed on the surface of E. coli cells. Redox equivalents are transferred from NADPH via the proteins AdR and Adx to the converting enzyme.

FIG. 18: NADPH consumption of cells by E. coli BL21(DE3) pETCYP13. Data points are the average of triplicate experiments. The bars represent the standard deviation. Squared symbols represent BL21(DE3) cells and round symbols represent BL21(DE3) pETCYP13 cells induced with 1 mM IPTG.

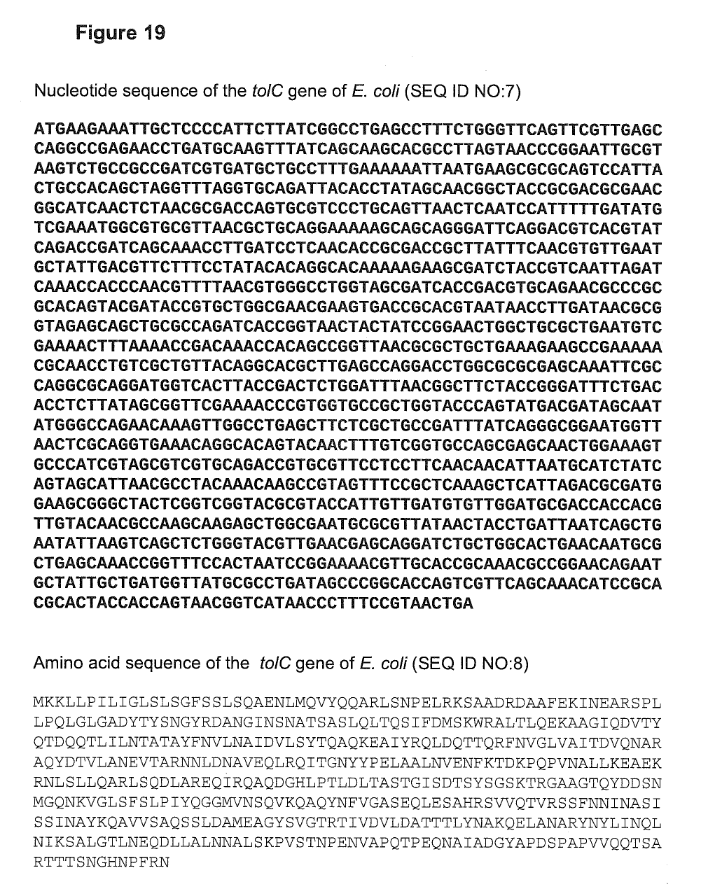

FIG. 19: Sequence of E. coli TolC. SEQ ID NO:7 describes a nucleotide sequence encoding TolC. SEQ ID NO:8 describes a TolC amino acid sequence.

### EXAMPLE 1

In this example, mainly two P450 enzymes shall be expressed by autodisplay, CYP106A2 and CYP3A4.

To establish an efficient HPLC analytic method experiments using the purified enzyme were conducted and retention times of the educt, product and internal standard determined. (FIG. 1a and FIG. 2) The gene of CYP106A2 was amplified by PCR and inserted into a plasmid encoding the domains needed for autodisplay. Successful expression was shown in an SDS-PAGE (FIG. 5) To find out whether the CYP106A2 domain of the fusion protein was indeed exposed at the cell surface, trypsin was added to whole cells of E. coli B121(DE3) pET-CYP13 after incubation with 1 mM IPTG for one hour. Trypsin is too large of a molecule to enter the cell envelope of E. coli. This means, if the CYP106A2 is degraded by trypsin, when added to whole cells, it must be accessible at the cell surface. Because OmpA, which has a N terminal extension in the periplasm, was not digested at all, it could be excluded, that trypsin had entered the periplasm due to cell leakyness. (FIG. 6a) Two additional methods to proof successful surface display came into operation as well; fluorescence microscopy (FIG. 6b) and FACS (FIG. 6c).

To measure if CYP106A4 was indeed expressed on the surface of the cells in a functional form activity tests were conducted. For that purpose cells were cultivated and protein expression induced using 1 mM Isopropyl-beta-D-thiogalactopyranosid (IPTG). In a conversion assay it was tested whether the cells displaying CYP106A2 on the surface have the ability to efficiently convert 11-deoxycorticosterone (DOC) into 15beta-DOC. To enhance the activity of the displayed enzyme adrenodoxin (Adx) and adrenodoxin reductase (AdR) from bovine adrenals, supplying this enzyme with the reducing equivalents necessary for steroid hydroxylation activity, were added. The use of whole E. coli cells only resulted in a product peak if protein expression was induced with 1 mM IPTG (FIG. 7). To proof that all conversions took only place on the surface of E. coli and not inside the cell by other electron supplying systems, substrate conversions were done without the addition of Adx, since it is too large of a molecule to enter the cell envelope. The chromatogram of this conversion assay shows, as expected, no product peak (FIG. 8).

Further experiments using a non-membrane transferrable substrate, abietic acid, were conducted. (FIGS. 9-12) and it was shown that a product occurred at the expected retention time if protein expression was induced with 1 mM IPTG.

A similar approach starting from amplification of the gene by PCR to successful HPLC activity measurements was performed for the human enzyme CYP3A4.

We succeeded to display the P450 enzymes CYP106A2 and CYP3A on the surface of E. coli in a functional form by using the autodisplay system. Functional expression was achieved by a one step procedure after induction of protein expression. Without wishing to by bound by theory, the prosthetic group was incorporated during transport in the periplasm, and the folded protein was translocated to the cell surface by the aid of the Omp85 pathway.

### EXAMPLE 2

This example refers to the role of TolC present in the outer membrane of E. coli cells displaying CYP106A2 on the surface. TolC is involved in porphyrin transport across the cell membrane [26].

Functional expression was determined by the CYP106A2-dependent conversion of 11-deoxycorticosterone to 15β-deoxycorticosterone, employing the experimental condition as described in Example 1.

The strain BL21 (DE3) employed in Example 1 expresses TolC on the outer membrane. Therefore, this strain has the capability to transport porphyrins, including P450, to the outer membrane surface.

The strain JW5503-1 (DE3) is a TolC defective mutant, with a reduced capability of transporting porphyrins (including P450) onto the outer membrane surface. JW5503-1 was obtained from the Keio collection distributed by Coli Genetic Stock Center at Yale University.

FIG. 15 indicates that CYP106A2 is expressed by pET-CYP13 to the same extent on the surface on TolC negative strain JW5503-1 (DE3) and on TolC positive BL21 (DE3) E. coli cells.

FIG. 16 shows HPLC chromatograms of CYP106A2 conversion of 11-deoxycorticosterone to 15β-deoxycorticosterone. By comparison of FIGS. 16C and B, it can be seen that the amount of 15β-deoxycorticosterone decreased when CYP106A2 was expressed in the E. coli strain lacking the TolC channel protein.

It is concluded that TolC can provide porphyrins, in particular P450, on the cell surface, so that the porphyrins can be introduced into a recombinant surface-displayed polypeptide of the present invention, such as CYP106A.

FIG. 16 indicates that there is still a CYP106A activity in the absence of TolC. Thus other transporters different from TolC may be present in the outer membrane so that porphyrins, in particular P450, can be provided on the cell surface so that the prosthetic group can be introduced into a recombinant surface-displayed polypeptide of the present invention, such as CYP106A.

### EXAMPLE 3

### N-Demethylation of the Antidepressant Imipramine into its Active Metabolite, Desipramine

The reaction as illustrated by FIG. 17 is catalyzed by CYP106A2 displayed on the surface of E. coli cells. The cells were prepared as described in Example 1. Redox equivalents are transferred from NADPH via the proteins AdR and Adx to the converting enzyme.

The reaction mixture contained in a total volume of 0.2 mL Hepes buffer (50 mM, 0.05% Tween20, pH 7.4), imipramine (2.5 μM), Adx (μM), AdR (0.5 μM), NADPH (200 μM) and cells of E. coli BL21(DE3) or BL21(DE3) pETCYP13 corresponding to an OD₅₇₈=2.5.

FIG. 18 shows the kinetics of NADPH consumption of cells by E. coli BL21(DE3) pETCYP13 expressing CYP106A2 cells and control BL21(DE3) cells in the presence of imipramine, as indicated above. The by-product formalin (cf. FIG. 17) has been photometrically identified to be produced by the cells displaying CYP106A2, but not in the control cells (data not shown). Thus, the difference of CYP106A2 expressing cells and control cells in NADPH consumption indicates conversion of imipramine into desipramine.

### REFERENCES

1. Kaur J, Sharma R (2006) Crit. Rev Biotechnol 26, 165-199.
2. Schoemaker H E, Mink D, Wubbolts M G (2003) Science 299, 1694-1697.
3. Rubin-Pitel S B, Zhao H (2006) Comb Chem High Throughput Screen 9, 247-257.
4. Bornscheuer U T (2005) Adv Biochem Engin Biotechnol 100, 181-203.
5. Bernhardt R (2006) J Biotechnol 124, 128-145.
6. Li Y, Drummond D A, Sawayama A M, Snow C D, Bloom J D, Arnold F H (2007) Nat Biotechnol 9, 1051-1056.
7. Hannemann F, Virus C, Bernhardt R (2006) J Biotechnol 124, 72-81.
8. Guengerich F P (2008) Chem Res Toxicol 21, 70-83.
9. Jose J, Meyer T F (2007) Microbiol Mol Biol 71, 600-619.
10. Jose J (2006) Appl Microbiol Biotechnol 69, 607-614.
11. Jose J, Bernhardt R, Hannemann F (2001) ChemBioChem 2, 695-701.
12. Jose J, Bernhardt R, Hannemann F (2002) J Biotechnol 95, 257-268.
13. Jose J, Jähnig F, Meyer T F (1995) Mol Microbiol 18, 380-382.
14. Pohlner J, Halter R, Beyreuther K, Meyer T F (1987) Nature 325, 458-462.
15. Maurer J, Jose J, Meyer T F (1997) J Bacteriol 179, 794-804.
16. Jose J, Handel S (2003) ChemBioChem 4, 396-405.
17. Jose J, von Schwichow S (2004) Anal Biochem 331, 267-274.
18. Klauser T, Pohlner J, Meyer T F (1990) Embo J 9, 1991-1999.
19. Jose J, Krämer J, Klauser T, Pohlner J, Meyer T F (1996) Gene 178, 107-110.
20. Jose J, Zangen D (2005) Biochem Biophys Res Commun 333, 1218-1226.
21. Jose J, von Schwichow S (2004) ChemBioChem 5, 491-499.
22. Hannemann F, Bernhardt R, Jose J (2007) J Enz Inhib Med Chem 22, 570-576.
23. Hantke, K., 1981. Regulation of ferric iron transport in Escherichia coli K12: isolation of a constitutive mutant. Mol. Gen. Genet. 182, 288-292.
24. Henderson I et al., 2004. Type V protein secretion pathway: the autotransporter story. Microbiology and Molecular Biology Reviews, 68(4), 692-744
25. Schultheiss, E., Paar, C., Schwab, H., Jose, J., 2002. Functional esterase surface display by the autotransporter pathway in Escherichia coli. J. Mol. Catal., B Enzym. 18, 89-97.
26. Ryoko Tatsumi, Masaaki Wachi (2008), TolC-Dependent Exclusion of Porphyrins in Escherichia coli. J Bacteriol. 190(18):6228-6233. Published online 2008 Jul. 18.

1. A method for displaying a recombinant polypeptide containing a prosthetic group on the surface of a host cell, wherein the prosthetic group comprises a metal porphyrin or a flavin, said method comprising the steps:
(a) providing a host cell transformed with a nucleic acid fusion operatively linked with an expression control sequence said nucleic acid fusion comprising:
(i) a portion encoding a signal peptide,
(ii) a portion encoding the recombinant polypeptide to be displayed,
(iii) optionally a portion encoding a protease recognition site,
(iv) a portion encoding a transmembrane linker, and
(v) a portion encoding the transporter domain of an autotransporter,
and
(b) culturing the host cell under conditions wherein the nucleic acid fusion is expressed and the expression product comprising the recombinant polypeptide containing the prosthetic group is displayed on the surface of the host cell.

2. The method according to claim 1, wherein the prosthetic group is transported to the cell surface independently from the expression product comprising the recombinant polypeptide.

3. The method according to claim 1 or 2 wherein metal porphyrin comprises one selected from cobalt, nickel, manganese, copper and iron.

4. The method according to any one of claims 1 to 3, wherein the metal porphyrin comprises a heme.

5. The method according to any one of the preceding claims wherein the polypeptide comprising the metal porphyrin is selected from hemoproteins, P450 enzymes, P450 reductases, cytochromes, and monooxygenases.

6. The method according to any one of the preceding claims, wherein the prosthetic group being a metal porphyrin is transported to the cell surface by a TolC-dependent mechanism.

7. The method according to claim 6, wherein the cell is a Gram-negative cell, and the prosthetic group is transported across the outer membrane by TolC.

8. The method according to claim 6 or 7, wherein the TolC is a recombinant TolC.

9. The method according to any one of the claims 6 to 8, wherein the TolC polypeptide is homologous to the host cell.

10. The method according to claim 1 wherein the polypeptide comprises a flavin selected from FAD and FMN.

11. The method according to claim 10 wherein the polypeptide comprising a flavin is selected from flavoproteins.

12. The method according to any one of the preceding claims wherein the host cell is a bacterium, particularly a Gram-negative bacterium, moreparticularly an enterobacterium, e.g. E. coli.

13. The method according to any one of the preceding claims wherein the transporter domain of the autotransporter forms a β-barrel structure.

14. The method according to any one of the preceding claims wherein the transporter domain of the autotransporter is selected from Ssp, Ssp-h1, Ssp-h2, PspA, PspB, Ssa1, SphB1, AspA/NalP, VacA, AIDA-I, IcsA, MisL, TibA, Ag43, ShdA, AutA, Tsh, SepA, EspC, EspP, Pet, Pic, SigA, Sat, Vat, EpeA, EatA, EspI, EaaA, EaaC, Pertactin, BrkA, Tef, Vag8, PmpD, Pmp20, Pmp21, IgA1 protease, App, Hap, rOmpA, rOmpB, ApeE, EstA, Lip-I, McaP, BabA, SabA, AlpA, Aae, NanB, and variants thereof.

15. The method according to any one of the preceding claims wherein the transporter domain of the autotransporter is the E. coli AIDA-I protein or a variant thereof.

16. The method according to any one of the preceding claims wherein in step (b), the prosthetic group endogenously produced in the cell is introduced into the recombinant polypeptide within the periplasmic space.

17. The method according to any one of the preceding claims wherein step (b) comprises transportation of the recombinant polypeptide via the omp85 pathway.

18. Host cell displaying a recombinant polypeptide on the surface thereof wherein the recombinant polypeptide contains a prosthetic group comprising a metal porphyrin or a flavin, and wherein the recombinant polypeptide comprises
(I) a portion comprising the recombinant polypeptide to be displayed,
(II) optionally a portion comprising a protease recognition site,
(III) a portion comprising a transmembrane linker, and
(IV) a portion comprising the transporter domain of an autotransporter.

19. The host cell of claim 18 wherein the recombinant polypeptide is displayed by the transporter domain of an autotransporter.

20. The host cell according to claim 18 or 19, wherein the prosthetic group is transported to the cell surface independently from the expression product comprising the recombinant polypeptide.

21. The host cell of any one of the claims 18 to 20, wherein metal porphyrin comprises one selected from cobalt, nickel, manganese, copper and iron.

22. The host cell according to any one of the claims 18 to 21, wherein the metal porphyrin comprises a heme.

23. The host cell according to any one of the claims 18 to 22, wherein the polypeptide comprising the metal porphyrin is selected from hemoproteins, P450 enzymes, P450 reductases, cytochromes, and monooxygenases.

24. The host cell according to any one of the claims 18 to 23, wherein the prosthetic group being a metal porphyrin is transported to the cell surface by a TolC-dependent mechanism.

25. The host cell according to claim 24, wherein the cell is a Gram-negative cell, and the prosthetic group is transported across the outer membrane by TolC.

26. The host cell according to claim 24 or 25, wherein the TolC is a recombinant TolC.

27. The host cell according to any one of the claims 24 to 26, wherein the TolC polypeptide is homologous to the host cell.

28. The host cell according to any one of the claims 18 to 20 wherein the polypeptide comprises a flavin selected from FAD and FMN.

29. The host cell according to any one of the claims 18 to 20 and 28 wherein the polypeptide comprising a flavin is selected from flavoproteins.

30. The host cell according to any one of the claims 18 to 29 wherein the host cell is a bacterium, particularly a Gram-negative bacterium, more particularly an enterobacterium, e.g. E. coli.

31. Membrane preparation which is derived from a host cell of any one of the claims 18 to 30, wherein the membrane preparation comprises in particular membrane particles.

32. Use of a cell of any one of the claims 18 to 30 or a membrane preparation of claim 31 for a chemical synthesis procedure.

33. Use of claim 32 for the synthesis of organic substances selected from enzyme substrates, drugs, hormones, starting materials and intermediates for synthesis procedures and chiral substances.

34. Use of a cell of any one of the claims 18 to 30 or a membrane preparation of claim 31 for a directed evolution procedure.

35. Use of a cell of any one of the claims 18 to 30 or a membrane preparation of claim 31 as an assay system for a screening procedure, e.g. for identifying modulators of metal porphyrin containing enzymes.

36. Use of a cell of any one of the claims 18 to 30 or a membrane preparation of claim 31 as a system for toxicity monitoring.

37. Use of a cell of any one of the claims 18 to 30 or a membrane preparation of claim 31 as a system for degrading toxic substances.


SEQUENCE LISTING (14 sequences)

SEQ ID NO: 1 - DNA, 1242 bp, Artificial Sequence
  CYP106A2 insert in plasmid pET-CYP13
ctcgagatggaagaagttattgcagtaaaagaaattactaggtttaaaacaaggacggaggaatttagcccgtacgcttggtgtaaaaggatgttagaaaatgaccctgtgagttatcacgaaggaacggatacgtggaatgtctttaaatatgaagatgtgaagcgggttctcagtgattataaacatttttcaagtgttcggaaacggacgacgatttcagttggaacggatagtgaggaaggttctgtgcctgaaaagatccaaatcactgaatcggatccacctgatcatagaaaacgccgttcactgctggcagcagcattcacacctagaagtcttcaaaactgggaacctcgcattcaggaaattgcagatgaattgattggacaaatggatggtggaacggaaatcgatattgtggcatcattggcgagtccgcttccgatcattgtcatggccgatttgatgggggttccctcgaaagatcgtttattgtttaagaaatgggtggataccttatttcttccttttgatagagaaaagcaagaagaagtagataaattgaagcaagttgcagcaaaagaatactatcagtatttgtatccgattgttgtgcaaaaacgattgaacccggcggatgatatcatctcagatctattgaagtcggaagtggatggggaaatgtttacggatgatgaggttgtccggacgaccatgctgattttaggtgcaggagtcgagacaaccagtcatttattggccaatagcttttattcgctgctatatgatgacaaagaagtttatcaagagttacatgaaaacctggatttagttccgcaggcggtcgaagaaatgctccgtttccgattcaatcttattaaattggatcgcactgtaaaggaagataacgatctattgggagtggaattgaaagaaggggatagcgtggttgtttggatgagtgcagctaatatggacgaagagatgtttgaagaccccttcacacttaatatccaccgccctaataataagaaacatctcacattcggtaatggccctcatttctgcctcggagcaccgctagccaggctggaagcgaagattgcgcttactgcattcctgaagaaattcaagcatattgaagcggtgccatcgttccagttagaagagaatcttaccgattcagcgaccggtcaaactttgacctcactaccgcttaaggcaagccgcatgggtacc

SEQ ID NO: 2 - protein, 414 aa, Artificial Sequence
  CYP106A2 insert in plasmid pET-CYP13
LEMEEVIAVKEITRFKTRTEEFSPYAWCKRMLENDPVSYHEGTDTWNVFKYEDVKRVLSDYKHFSSVRKRTTISVGTDSEEGSVPEKIQITESDPPDHRKRRSLLAAAFTPRSLQNWEPRIQEIADELIGQMDGGTEIDIVASLASPLPIIVMADLMGVPSKDRLLFKKWVDTLFLPFDREKQEEVDKLKQVAAKEYYQYLYPIVVQKRLNPADDIISDLLKSEVDGEMFTDDEVVRTTMLILGAGVETTSHLLANSFYSLLYDDKEVYQELHENLDLVPQAVEEMLRFRFNLIKLDRTVKEDNDLLGVELKEGDSVVVWMSAANMDEEMFEDPFTLNIHRPNNKKHLTFGNGPHFCLGAPLARLEAKIALTAFLKKFKHIEAVPSFQLEENLTDSATGQTLTSLPLKASRMGT

SEQ ID NO: 3 - protein, 810 aa, Escherichia coli
MAMKKLLIASLLFSSATVYGAEGFVVKDIHFEGLQRVAVGAALLSMPVRTGDTVNDEDISNTIRALFATGNFEDVRVLRDGDTLLVQVKERPTIASITFSGNKSVKDDMLKQNLEASGVRVGESLDRTTIADIEKGLEDFYYSVGKYSASVKAVVTPLPRNRVDLKLVFQEGVSAEIQQINIVGNHAFTTDELISHFQLRDEVPWWNVVGDRKYQKQKLAGDLETLRSYYLDRGYARFNIDSTQVSLTPDKKGIYVTVNITEGDQYKLSGVEVSGNLAGHSAEIEQLTKIEPGELYNGTKVTKMEDDIKKLLGRYGYAYPRVQSMPEINDADKTVKLRVNVDAGNRFYVRKIRFEGNDTSKDAVLRREMRQMEGAWLGSDLVDQGKERLNRLGFFETVDTDTQRVPGSPDQVDVVYKVKERNTGSFNFGIGYGTESGVSFQAGVQQDNWLGTGYAVGINGTKNDYQTYAELSVTNPYFTVDGVSLGGRLFYNDFQADDADLSDYTNKSYGTDVTLGFPINEYNSLRAGLGYVHNSLSNMQPQVAMWRYLYSMGEHPSTSDQDNSFKTDDFTFNYGWTYNKLDRGYFPTDGSRVNLTGKVTIPGSDNEYYKVTLDTATYVPIDDDHKWVVLGRTRWGYGDGLGGKEMPFYENFYAGGSSTVRGFQSNTIGPKAVYFPHQASNYDPDYDYECATQDGAKDLCKSDDAVGGNAMAVASLEFITPTPFISDKYANSVRTSFFWDMGTVWDTNWDSSQYSGYPDYSDPSNIRMSAGIALQWMSPLGPLVFSYAQPFKKYDGDKAEQFQFNIGKTW

SEQ ID NO: 4 - protein, 810 aa, Escherichia coli; identical to SEQ ID NO: 3

SEQ ID NO: 5 - DNA, 1491 bp, Artificial Sequence
  CYP3A4 in plasmid pSC001
ctcgagatggctctgttattagcagtttttctggtgctcctctatctatatggaacccattcacatggactttttaagaagcttggaattccagggcccacacctctgccttttttgggaaatattttgtcctaccataagggcttttgtatgtttgacatggaatgtcataaaaagtatggaaaagtgtggggcttttatgatggtcaacagcctgtgctggctatcacagatcctgacatgatcaaaacagtgctagtgaaagaatgttattctgtcttcacaaaccggaggccttttggtccagtgggatttatgaaaagtgccatctctatagctgaggatgaagaatggaagagattacgatcattgctgtctccaaccttcaccagtggaaaactcaaggagatggtccctatcattgcccagtatggagatgtgttggtgagaaatctgaggcgggaagcagagacaggcaagcctgtcaccttgaaagacgtctttggggcctacagcatggatgtgatcactagcacatcatttggagtgaacatcgactctctcaacaatccacaagacccctttgtggaaaacaccaagaagcttttaagatttgattttttggatccattctttctctcaataacagtctttccattcctcatcccaattcttgaagtattaaatatctgtgtgtttccaagagaagttacaaattttttaagaaaatctgtaaaaaggatgaaagaaagtcgcctcgaagatacacaaaagcaccgagtggatttccttcagctgatgattgactctcagaattcaaaagaaactgagtcccacaaagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggcaccacccacctatgatactgtgctacagatggagtatcttgacatggtggtgaatgaaacgctcagattattcccaattgctatgagacttgagagggtctgcaaaaaagatgttgagatcaatgggatgttcattcccaaaggggtggtggtgatgattccaagctatgctcttcaccgtgacccaaagtactggacagagcctgagaagttcctccctgaaagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacagatccccctgaaattaagcttaggaggacttcttcaaccagaaaaacccgttgttctaaaggttgagtcaagggatggcaccgtaagtggagccggtacc

SEQ ID NO: 6 - protein, 497 aa, Artificial Sequence
  Amino acid sequence of CYP3A4 in plasmid pSC001
LEMALLLAVFLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGAGT

SEQ ID NO: 7 - DNA, 1482 bp, Escherichia coli
atgaagaaattgctccccattcttatcggcctgagcctttctgggttcagttcgttgagccaggccgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccctgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 8 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 9 - DNA, 93 bp, Artificial Sequence
  Fragment of a typical artificial autotransporter
ctatcttcagcatatgcacatggaacaccttctagactcgagagatcttgccctgaatatttcaaaggtccaccttctccacgatctcttaat

SEQ ID NO: 10 - protein, 31 aa, Artificial Sequence
  Fragment of a typical artificial autotransporter
LSSAYAHGTPSRLERSCPEYFKGPPSPRSLN

SEQ ID NO: 11 - DNA, 24 bp, Artificial Sequence
  Fragment with important restriction site
actgatttgctcgagatggaagaa

SEQ ID NO: 12 - protein, 8 aa, Artificial Sequence
  Fragment with important restriction site
TDLLEMEE

SEQ ID NO: 13 - DNA, 24 bp, Artificial Sequence
  Fragment with important restriction site
agccgcatgggtacccttaatcct

SEQ ID NO: 14 - protein, 8 aa, Artificial Sequence
  Fragment with important restriction site
SRMGTLNP